(19) United States
(12) Patent Application Publication
(10) Pub. No.: US 2016/0122768 A1
(43) Pub. Date: May 5, 2016

(54) AUTOIMMUNE DISEASE TREATMENTS

(71) Applicant: LA JOLLA INSTITUTE FOR ALLERGY AND IMMUNOLOGY, La Jolla, CA (US)

(72) Inventors: Nunzio Bottini, San Diego, CA (US); Stephanie Stanford, La Jolla, CA (US)

(21) Appl. No.: 14/937,439

(22) Filed: Nov. 10, 2015

Related U.S. Application Data
(63) Continuation of application No. PCT/US2014/037539, filed on May 9, 2014.
(60) Provisional application No. 61/822,155, filed on May 10, 2013.

(51) Int. Cl. C12N 15/113; A61K 38/00; C07K 16/40

(52) U.S. Cl. CPC C12N 15/1137; C07K 16/40; A61K 38/00; C12N 2310/11; C12N 2310/14

(57) ABSTRACT

There are provided, inter alia, methods and compositions to treat autoimmune disease including invasiveness of fibroblast-like synoviocytes in rheumatoid arthritis.

### CROSS-REFERENCES TO RELATED APPLICATIONS

This application is a continuation of International Application No. PCT/US2014/037539, filed May 9, 2014, which claims the benefit of U.S. Provisional Application No. 61/822,155, filed May 10, 2013, the content of each of which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 48513-505C01US_ST25.TXT, created Nov. 4, 2015, 17,270 bytes, machine format IBM-PC, MS-Windows operating system, is hereby incorporated herein by reference in its entirety and for all purposes.

### BACKGROUND OF THE INVENTION

Invasiveness is a pathogenic phenotype of fibroblast-like synoviocytes (FLS) in rheumatoid arthritis (RA). See e.g., Noss, E. H., and Brenner, M. B., 2008. Immunological reviews, 223:252-270; Bottini, N., & Firestein, G. S. 2013. Nat Rev Rheumatol, 9:24-33). FLS secrete components of synovial fluid and provide structural and dynamic support to the joint. In rheumatoid arthritis (RA) however, FLS assume intrinsic invasive features, and mediate destruction of cartilage and bone. For example, FLS are an abundant source of IL-6 in the joints of subjects suffering RA. FLS obtained from patients with RA and cultured ex vivo or implanted into immunodeficient mice display increased invasiveness compared to FLS from healthy subjects or patients with osteoarthritis (OA). See e.g., Bottini, N., & Firestein, G. S. 2013. Id. Targeting of FLS is being considered as an option for development of new therapies for RA. See e.g., Noss, E. H., and Brenner, M. B., 2008, Id.

Key proteins expressed by FLS include surface proteins, e.g., integrins, ICAM-1, VCAM-1, Cadherin-11, CD55, and CD90, intracellular proteins, e.g., vimentin, 6PGL, and collagen proteins, e.g., Type IV collagen and Type V collagen, as known in the art.

FLS behavior is controlled by a network of intracellular signaling pathways, many of which rely upon reversible phosphorylation of proteins on tyrosine residues. See e.g., Bottini, N., and Firestein, G. S. 2013, Id. Tyrosine phosphorylation results from the balanced action of protein tyrosine kinases (PTKs), which catalyze addition of phosphates on tyrosine residues, and phosphatases (PTPs), which counter that action by tyrosine dephosphorylation. At least 50 PTPs are expressed in FLS (Stanford, S. M. et al., 2013. Arthritis Rheum, 65:1171-1180), however little is known about the involvement of PTPs in FLS functions. To identify signaling mediators involved in promoting the unique aggressive phenotype of RA FLS, we explored the role of a PTP overexpressed in RA compared to OA FLS, PTPRK (receptor tyrosine-protein phosphatase kappa).

There are provided herein, inter alia, methods and compositions for treatment of autoimmune disease including invasiveness of FLS in RA.

### BRIEF SUMMARY OF THE INVENTION

In a first aspect, there is provided a method of treating an autoimmune disease in a subject in need thereof, the method including administering to the subject an effective amount of a PTPRK antagonist.

In another aspect, there is provided a method of decreasing inflammation in a synovium of a subject in need thereof, the method including administering to the subject an effective amount of a PTPRK antagonist.

In another aspect, there is provided a method of decreasing expression of PTPRK in a fibroblast-like synoviocyte, the method including contacting said fibroblast-like synoviocyte with an effective amount of a PTPRK antagonist.

In another aspect, there is provided a decreasing TNF activity, IL-1 activity or PDGF activity in a fibroblast-like synoviocyte, the method including contacting the fibroblast-like synoviocyte with an effective amount of a PTPRK antagonist.

In another aspect, there is provided a method of decreasing invasiveness or migration of a fibroblast-like synoviocyte, the method including contacting the fibroblast-like synoviocyte with an effective amount of a PTPRK antagonist.

In another aspect, there is provided a pharmaceutical composition including a PTPRK antagonist and a pharmaceutically acceptable excipient.

### BRIEF DESCRIPTION OF THE DRAWINGS

FIGS. 1A-1F. TGFβ1-responsive PTPRK is required for RA FLS invasiveness. FIG. 1A: Histogram depicts results of PTPRK mRNA expression in FLS measured by qPCR. Median±interquartile range (IQR) is shown. *, p<0.05, Mann-Whitney test. Legend (left to right) OA FLS (n=12); RA FLS (n=13). FIG. 1B: Western blotting of lysates from 3 RA and 3 OA FLS lines. FIG. 1C: Following treatment with control (Ctl) or PTPRK ASO for 7 d, RA FLS invaded through Matrigel-coated transwell chambers in response to 50 ng/ml PDGF-BB for 24 hr. Graph shows median±IQR % maximum number of cells per field. *, p<0.05, Mann-Whitney test. Data from 4 independent experiments in different FLS lines is shown. FIG. 1D: ASO-treated RA FLS migrated through uncoated transwell chambers in response to 5% FBS for 24 hr. Graph shows median±IQR % maximum number of cells per field. Data from 5 independent experiments in different FLS lines is shown. *, p<0.05, Mann-Whitney test. FIG. 1E: ASO-treated RA FLS migrated out of a Matrigel sphere for 2 d in response to 10 ng/ml PDGF or media alone. Left panel, graph shows mean±SD cells per field. Data from 3 independent experiments in different FLS lines is shown. Significance was calculated using the paired t-test. *, p<0.05. Right panel, representative image. FIG. 1F: ASO-treated RA FLS were plated on fibronectin (FN)-coated coverslips in the presence of 5% FBS. Graphs show median±IQR cell area after 15, 30 and 60 min. Data from 3 independent experiments in different FLS lines is shown. *, p<0.05, Mann-Whitney test.

FIGS. 2A-2B. RPTPκ promotes RA FLS migration through dephosphorylation of SRC. FIGS. 2A-2B: RA FLS migrated through uncoated transwell chambers in response to 5% FBS in the presence of the SRC family kinase inhibitor PP2 (FIG. 2A) or the phospholipase C gamma 1 inhibitor U73122 (FIG. 2B). Dimethyl sulfoxide (DMSO) is a control. Histograms depict median±IQR % maximum number of cells per field. Data from 2 independent experiments in different FLS lines is shown. *, p<0.05, Mann-Whitney test.

FIGS. 3A-3I. RPTPκ is required for the pathogenic action of RA FLS. FIGS. 3A-3E depict histograms of results of ASO-treated RA FLS which were stimulated with 50 ng/ml TNFα for 24 hr or left unstimulated. Graph shows mean±SD relative mRNA expression levels. *, p<0.05, paired t-test. For each of FIGS. 3A-3E, left histogram group is unstimulated, and right histogram group is TNFα stimulated; for each group, ordering is control ASO (Ctl ASO) and PTPRK ASO, left to right. Legend: FIGS. 3A-3E: CXCL10 mRNA, VCAM1 mRNA, MMP2 mRNA, MMP8 mRNA and MMP13 mRNA, respectively. FIG. 3F: ASO-treated RA FLS were intradermally implanted into nude mice following subcutaneous injection of CFA. After 5 d, FLS invasion towards the inflammation site was measured by immunohistochemical staining of FLS in skin immediately adjacent the CFA injection site. Graph shows median±IQR cells per field. Data from 3 independent experiments in different FLS lines is shown. *, p<0.05, Mann-Whitney test. Legend: Left histogram group (Ctl ASO); right histogram group (PTPRK ASO). FIG. 3G: Model depicting role of TGFβ-dependent RPTPκ in RA pathogenesis. FIG. 3H-3I: To induce PTPRK expression, ASO-treated RA FLS were prestimulated with 50 ng/ml TGFβ1 for 24 hr (or left unstimulated) in the presence of ASO. Cells were then stimulated with 50 ng/ml TGFβ1/PDGF/TNFα, or 50 ng/ml PDGF/TNFα, or left unstimulated for 24 hr. Graph shows mean±SD relative mRNA expression levels for MMP13 mRNA (FIG. 3H) and MMP14 mRNA (FIG. 3I). *, p<0.05, paired t-test. Legend: FIGS. 3H-3I: left histogram group is unstimulated, middle histogram group is PDGF/TNFα stimulated, and right histogram group is TGFβ1/PDGF/TNFα stimulated. For each group, ordering is control ASO (Ctl ASO) and PTPRK ASO, left to right.

FIG. 4. Following treatment with 2.5 μM Ctl or PTPRK ASO for 7 days, RA FLS were stained with anti-beta-catenin antibody, phalloidin and Hoechst and imaged by immunofluorescence microscopy. Histogram FIG. 4 depicts proportions of beta-catenin in cytosolic and nuclear fractions. Data from 3 independent experiments in different FLS lines is shown. Significance was calculated using the Mann-Whitney test. Legend: left group (cytosol); right group (nucleus). Within each group the ASO employed was control ASO (Ctl ASO) and PTPRK ASO, in order left to right.

FIG. 5. RPTPκ is expressed in RA synovial lining. Panels show representative immunohistochemical staining of RA synovial sections using anti-RPTPκ (left panels) or control IgG antibodies (rights panels).

FIGS. 6A-6B. Knockdown of RPTPκ, but not RPTPμ, reduces RA FLS invasiveness. FIG. 6A: PTPRK_2 ASO enables efficient knockdown of RPTPκ. RA FLS were treated with 2.5 μM Ctl or PTPRK_2 ASO for 7 days. PTPRK expression was assessed by qPCR, normalized to the housekeeping gene RPII, and plotted relative to the PTPRK expression in Ctl ASO-treated cells. Panel shows mean±range. Data from 2 independent experiments in different FLS lines is shown. Legend (histogram, left to right): Control ASO (Ctl ASO) (SEQ ID N0:6); PTPRK_2 ASO (SEQ ID N0:4). FIG. 6B: Following treatment with 2.5 μM Ctl or PTPRK_2 ASO for 7 d, RA FLS invaded through Matrigel-coated transwell chambers in response to 50 ng/ml PDGF-BB for 24 hr. Graph shows median±IQR % maximum number of cells per field. Data from 3 independent experiments in different FLS lines is shown. Significance was calculated using the Mann-Whitney test, *, p<0.05. 0.05. Legend: see FIG. 6A.

FIGS. 7A-7G. RPTPκ promotes IL-1 signaling in RA FLS. Following treatment with Ctl ASO or PTPRK ASO for 7 days, RA FLS were stimulated with 50 ng/ml IL-1β for 24 h or left unstimulated. FIGS. 7A-7E depict histograms of data for CXC10 mRNA, VCAM1 mRNA, MMP2 mRNA, MMP3 mRNA, and MMP13 mRNA, respectively. These histograms depict mean±SD relative mRNA expression levels. For each of FIGS. 7A-7E, histogram entries are (left to right): unstimulated FLS with Ctrl ASO, unstimulated FLS with PTPRK ASO, IL-1 stimulated FLS with Ctl ASO, and IL-1 stimulated FLS with PTPRK ASO. Significance was calculated using the paired t-test *, p<0.05. FIGS. 7F-7G: Representative 40× images of mouse skin samples from experiment described in Example 5 (e.g., FIG. 3C), showing results from administration of Ctl ASO (FIG. 7F) and PTPRK ASO (FIG. 7G). Arrows indicate invading FLS stained with an anti human Class I HLA antibody.

### DETAILED DESCRIPTION OF THE INVENTION

### I. Definitions

The terms “PTPR,” “RPTP,” “rPTP” and the like refer, in the usual and customary sense, to receptor-type protein tyrosine phosphatases, which are found in nature as membrane bound protein tyrosine phosphatases. In embodiments, the RPTP is a mammalian RPTP (e.g. human, mouse, rat, or other mammal). In embodiments, the RPTP is a human RPTP. In embodiments, the RPTP refers to the protein encoded by the gene PTPRK. It is understood that the term “PTPRK” in the context of a gene refers to the gene encoding receptor tyrosine-protein phosphatase kappa. It is further understood that the terms “PTPRK,” “RPTPκ,” “RPTPk” and the like in the context of a protein refer to receptor tyrosine-protein phosphatase kappa. In embodiments, RPTP means the full length RPTP (e.g. the protein translated from the complete coding region of the gene, which may also include post-translational modifications). In embodiments RPTP includes a fragment of the RPTP full length protein or a functional fragment of the full length RPTP protein. In embodiments this definition includes one or all splice variants of an RPTP. An RPTP may include all homologs of the RPTP. In embodiments, PTPRK refers to mammalian PTPRK. In embodiments, a PTPRK refers to a human PTPRK. In embodiments, an RPTP includes all splice variants of the RPTP. In embodiments, an RPTP may refer to 1, 2, 3, 4, 5, 6, 7, 8, 9, 10, or more splice variants.

The terms “PTPR antagonist,” “RPTP antagonist” and the like refer to an agent which reduces the level of activity or a PTPR or the level of expression of a PTPR, e.g., RPTPk. The term “PTPRK antagonist” refers to an agent which reduces the level of activity or the level of expression of RPTPκ. A PTPR antagonist can be a RPTP binding agent, a RPTP small molecule inhibitor, a RPTP allosteric inhibitor, an anti-PTPR antibody, an anti-PTPR inhibitory nucleic acid, an anti-PTPR RNAi molecule, or a PTPR ligand mimetic, as disclosed herein. An “RPTP small molecule inhibitor” is a small molecule which inhibits an RPTP. An “PTPRK small molecule inhibitor” is a small molecule which inhibits PTPRK. An “RPTP allosteric inhibitor” is a molecule which can bind an allosteric site of an RPTP, thereby inhibiting the RPTP. An “PTPRK allosteric inhibitor” is a molecule which can bind an allosteric site of PTPRK, thereby inhibiting the PTPRK. RPTP allosteric inhibitors (e.g., PTPRK allosteric inhibitors) can bind either to the intracellular or extracellular regions of the RPTP (e.g., PTPRK) in regions of the protein that are not necessarily binding sites for other ligands, as known in the art. In embodiments, these agents do not directly compete for binding with a ligand, but could cause a change in the conformation of the protein that would affect either its binding to ligands, its activity, or other functions as known in the art.

The terms “subject,” “patient,” “individual,” etc. are not intended to be limiting and can be generally interchanged. That is, an individual described as a “patient” does not necessarily have a given disease, but may be merely seeking medical advice.

A “standard control” refers to a sample, measurement, or value that serves as a reference, usually a known reference, for comparison to a test sample, measurement, or value. For example, a test sample can be taken from a patient suspected of having a given disease (e.g. an autoimmune disease, inflammatory autoimmune disease, cancer, infectious disease, immune disease, or other disease) and compared to a known normal (i.e., non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. One of skill will recognize that standard controls can be designed for assessment of any number of parameters (e.g. RNA levels, protein levels, individual RPTP levels, specific cell types, specific bodily fluids, specific tissues, synoviocytes, synovial fluid, synovial tissue, fibroblast-like synoviocytes, macrophage-like synoviocytes, and the like).

One of skill in the art will understand which standard controls are most appropriate in a given situation and be able to analyze data based on comparisons to standard control values. Standard controls are also valuable for determining the significance (e.g. statistical significance) of data, as known in the art.

The terms “dose” and “dosage” are used interchangeably herein. A dose refers to the amount of active ingredient given to an individual at each administration, or to an amount administered in vitro or ex vivo. For the methods and compositions provided herein, the dose may generally depend to the required treatment for the disease (e.g. an autoimmune, inflammatory autoimmune, cancer, infectious, immune, or other disease), and the biological activity of the RPTP binding agent, RPTP antagonist, anti-PTPR antibody, anti-PTPR inhibitory nucleic acid, anti-PTPR RNAi molecule, or PTPR ligand mimetic. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

By “effective amount,” “therapeutically effective amount,” “therapeutically effective dose or amount” and the like as used herein is meant an amount (e.g., a dose) that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “diagnosis” refers to a relative probability that a disease (e.g. an autoimmune, inflammatory autoimmune, cancer, infectious, immune, or other disease) is present in the subject. The term “prognosis” refers to a relative probability that a certain future outcome may occur in the subject with respect to a disease state. For example, in the present context, prognosis can refer to the likelihood that an individual will develop a disease (e.g. an autoimmune, inflammatory autoimmune, cancer, infectious, immune, or other disease), or the likely severity of the disease (e.g., extent of pathological effect and duration of disease). The terms are not intended to be absolute, as will be appreciated by any one of skill in the field of medical diagnostics.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, or even longer. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, a method using high affinity interactions may achieve the same results where one of a pair of binding partners binds to the other, e.g., biotin, streptavidin.

The terms “identical” or percent sequence “identity,” in the context of two or more nucleic acids or polypeptide sequences, refer to two or more sequences or subsequences that are the same or have a specified percentage of amino acid residues or nucleotides that are the same (i.e., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a nucleic acid will hybridize to its target sequence, typically in a complex mixture of nucleic acids, but only weakly to other (i.e., non-target) sequences (e.g., 10-fold less, 100-fold less, 1000-fold less, or even less affinity) or not at all (i.e., no detectable hybridization to a sequence which is not a target sequence). Stringent conditions are sequence-dependent and will be different in different circumstances. Longer sequences hybridize specifically at higher temperatures. An extensive guide to the hybridization of nucleic acids is found in Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Generally, stringent hybridization conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

Nucleic acids may be substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

An “inhibitory nucleic acid” is a nucleic acid (e.g. DNA, RNA, polymer of nucleotide analogs) that is capable of binding to a target nucleic acid (e.g. an mRNA translatable into an RPTP) and reducing transcription of the target nucleic acid (e.g. mRNA from DNA) or reducing the translation of the target nucleic acid (e.g., mRNA) or altering transcript splicing (e.g. single stranded morpholino oligo). A “morpholino oligo” may be alternatively referred to as a “morpholino nucleic acid” and refers to morpholine-containing nucleic acid nucleic acids commonly known in the art (e.g. phosphoramidate morpholinio oligo or a “PMO”). See Marcos, P., Biochemical and Biophysical Research Communications 358 (2007) 521-527. In embodiments, the “inhibitory nucleic acid” is a nucleic acid that is capable of binding (e.g. hybridizing) to a target nucleic acid (e.g. an mRNA translatable into an RPTP) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence. An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In embodiments, the inhibitory nucleic acid is single stranded. In embodiments, the inhibitory nucleic acid is double stranded.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In embodiments, the antisense nucleic acid hybridizes to the target nucleic acid sequence (e.g. mRNA) under stringent hybridization conditions. In embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides. An “anti-PTPR antisense nucleic acid” is an antisense nucleic acid that is at least partially complementary to at least a portion of a target nucleic acid sequence, such as an mRNA molecule, that codes at least a portion of the PTPR. An “PTPRK antisense nucleic acid” is an antisense nucleic acid that is at least partially complementary to at least a portion of a target nucleic acid sequence, such as an mRNA molecule, that codes at least a portion of RPTPk.

In embodiments, an antisense nucleic acid is a morpholino oligo. In embodiments, a morpholino oligo is a single stranded antisense nucleic acid, as is know in the art. In embodiments, a morpholino oligo decreases protein expression of a target, reduces translation of the target mRNA, reduces translation initiation of the target mRNA, or modifies transcript splicing. In embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate a mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

Inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art Inhibitory nucleic acids (e.g. antisense nucleic acids, morpholino oligos) may be delivered to a cell using cell permeable delivery systems (e.g. cell permeable peptides). In embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

Construction of suitable vectors containing the desired therapeutic gene coding and control sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods disclosed herein. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), the size and type of a tumor (i.e., solid or suspended (i.e., blood or ascites)), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, BIOCONJUGATE TECHNIQUES 1996, Academic Press, Inc., San Diego.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In embodiments, antibodies or fragments of antibodies may be derived from different organisms, including humans, mice, rats, hamsters, camels, etc. Antibodies disclosed herein may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies as disclosed herein and for use according to the methods disclosed herein, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in MONOCLONAL ANTIBODIES AND CANCER THERAPY, Alan R. Liss, Inc. (1985); Coligan, CURRENT PROTOCOLS IN IMMUNOLOGY (1991); Harlow & Lane, ANTIBODIES, A LABORATORY MANUAL (1988); and Goding, MONOCLONAL ANTIBODIES: PRINCIPLES AND PRACTICE (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, IMMUNOLOGY (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides as disclosed herein. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the present disclosure include humanized and/or chimeric monoclonal antibodies.

In embodiments, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein. Such effector moieties include, but are not limited to, an anti-tumor drug, a toxin, a radioactive agent, a cytokine, a second antibody or an enzyme.

The immunoconjugate can be used for targeting the effector moiety to an RPTPk positive cell, i.e., cells which express RPTPk, assay of which can be readily apparent when viewing the bands of gels with approximately similarly loaded with test and controls samples. Examples of cytotoxic agents include, but are not limited to ricin, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, and glucocorticoid and other chemotherapeutic agents, as well as radioisotopes. Suitable detectable markers include, but are not limited to, a radioisotope, a fluorescent compound, a bioluminescent compound, chemiluminescent compound, a metal chelator or an enzyme.

Additionally, the recombinant proteins disclosed herein including the antigen-binding region of any of the antibodies disclosed herein can be used to treat inflammation. In such a situation, the antigen-binding region of the recombinant protein is joined to at least a functionally active portion of a second protein having therapeutic activity. The second protein can include, but is not limited to, an enzyme, lymphokine, oncostatin or toxin. Suitable toxins include doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, ricin, abrin, glucocorticoid and radioisotopes.

Techniques for conjugating therapeutic agents to antibodies are well known (see, e.g., Amon et al., “Monoclonal Antibodies For Immunotargeting Of Drugs In Cancer Therapy”, in MONOCLONAL ANTIBODIES AND CANCER THERAPY, Reisfeld et al. (eds.), pp. 243-56 (Alan R. Liss, Inc. 1985); Hellstrom et al., ANTIBODIES FOR DRUG DELIVERY IN CONTROLLED DRUG DELIVERY (2nd Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “pharmaceutically acceptable” is used synonymously with “physiologically acceptable” and “pharmacologically acceptable”. A pharmaceutical composition will generally include agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and/or absorption by a subject and can be included in the compositions disclosed herein without causing a significant adverse toxicological effect on the patient. Unless indicated to the contrary, the terms “active agent,” “active ingredient,” “therapeutically active agent,” “therapeutic agent” and like are used synonymously. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, polyethylene glycol, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the methods and compositions disclosed herein.

Certain compounds disclosed herein can exist in unsolvated forms as well as solvated forms, including hydrated forms. In general, the solvated forms are equivalent to unsolvated forms and are intended to be encompassed within the scope of the present disclosure. Certain compounds disclosed herein may exist in multiple crystalline or amorphous forms. In general, all physical forms are equivalent for the uses contemplated herein and are intended to be within the scope of the present disclosure.

A “protein level of an RPTP” refers to an amount (relative or absolute) of RPTP in its protein form (as distinguished from its precursor RNA form). A protein of an RPTP may include a full-length protein (e.g. the protein translated from the complete coding region of the gene, which may also include post-translational modifications), functional fragments of the full length protein (e.g. sub-domains of the full length protein that possess an activity or function in an assay), or protein fragments of the RPTP, which may be any peptide or oligopeptide of the full length protein.

An “RNA level of an RPTP” refers to an amount (relative or absolute) of RNA present that may be translated to form an RPTP. The RNA of an RPTP may be a full-length RNA sufficient to form a full-length RPTP. The RNA of an RPTP may also be a fragment of the full length RNA thereby forming a fragment of the full length RPTP. The fragment of the full length RNA may form a functional fragment of the RPTP. In embodiments, the RNA of an RPTP includes all splice variants of an RPTPR gene.

An “autoimmune therapeutic agent” is a molecule (e.g. antibody, nucleic acid, inhibitory nucleic acid, ligand mimetic, small chemical molecule) that treats or prevents an autoimmune disease when administered to a subject in a therapeutically effective dose or amount. In embodiments, an autoimmune therapeutic agent is an RPTP binding agent. In embodiments, the therapeutic agent can bind to more than one RPTP.

An “IAD therapeutic agent” is a molecule that treats or prevents an inflammatory autoimmune disease (IAD) when administered to a subject in a therapeutically effective dose or amount where the autoimmune disease is mediated by a PTPR. Some non-limiting examples of an IAD therapeutic agent include an IAD PTPR binding agent, anti-IAD PTPR antibody, anti-IAD PTPR inhibitory nucleic acid, anti-PTPRK RNAi molecule, and an IAD PTPR ligand mimetic. In embodiments, IAD therapeutic agents are useful in methods and compositions described herein relating to any autoimmune disease. In embodiments, the IAD therapeutic agent can bind to more than one RPTP. In embodiments, the IAD therapeutic agent can bind to RPTPk.

An “RPTP binding agent” is a molecule that binds (e.g. preferentially binds) to one or more RPTPs, RNA that is translatable to an RPTP, or DNA that is transcribable to an RNA that is translatable to an RPTP. Where the molecule preferentially binds, the binding is preferential as compared to other macromolecular biomolecules present in an organism or cell. A compound preferentially binds to as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In embodiments, the RPTP binding agent preferentially binds to one or more RPTPs. In embodiments, the RPTP binding agent preferentially binds to one RPTP (e.g. RPTPk) in comparison to one or more other RPTPs. In embodiments, the RPTP binding agent preferentially binds to an RNA that is translatable to an RPTP (e.g. RPTPk) compared to an RNA that is translatable to another RPTP nucleic acids. In embodiments, the RNA is mRNA. In embodiments, the RPTP binding agent is a protein, nucleic acid, ligand, ligand mimetic, or a small chemical molecule. In embodiments, an RPTP binding agent disrupts the interaction between an RPTP and a physiological or natural ligand. In embodiments, an RPTP binding agent binds a physiological or natural ligand of the RPTP. In embodiments, an RPTP binding agent binds the complex of an RPTP bound to a ligand. In embodiments, the binding agent can bind to more than one RPTP. An “RPTPk binding agent” or “PTPRK binding agent” is an RPTP binding agent that binds RPTPk.

An “anti-PTPR antibody” is an antibody, as disclosed herein and well known in the art, directed to a PTPR. The term “anti-PTPRK antibody” and the like refer to an antibody directed to RPTPk.

An “anti-PTPR inhibitory nucleic acid” is an inhibitory nucleic acid that is capable of hybridizing to target nucleic acid sequence (e.g. an mRNA sequence) that is translatable to a PTPR (e.g., RPTPk) or a target nucleic acid sequence (e.g. a DNA sequence) that is transcribable to an RNA that is translatable to a PTPR. The anti-PTPR inhibitory nucleic acid is typically capable of decreasing the amount of PTPR that is translated in a cell. An “anti-PTPRK inhibitory nucleic acid” is an inhibitory nucleic acid that is capable of hybridizing to target nucleic acid sequence (e.g. an mRNA sequence) that is translatable to RPTPk or a target nucleic acid sequence (e.g. a DNA sequence) that is transcribable to an RNA that is translatable to RPTPk.

An “anti-PTPR RNAi molecule” is an siRNA, shRNA, miRNA, shmiRNA, or other nucleic acid, as well known in the art, that is capable of inducing RNAi and hybridizing to an RNA that is translatable to a PTPR. The anti-PTPR RNAi molecule is typically capable of decreasing the amount of PTPR that is translated in a cell. An “anti-PTPRK RNAi molecule” is an siRNA, shRNA, miRNA, shmiRNA, or other nucleic acid, as well known in the art, that is capable of inducing RNAi and hybridizing to an RNA that is translatable to a RPTPk.

An “PTPR ligand mimetic” is a PTPR binding agent that is designed to mimic, in structure or in binding mode, a PTPR ligand or is capable of inhibiting the binding of a natural or physiological ligand to an PTPR. In embodiments, a PTPR ligand mimetic is a synthetic chemical compound, peptide, protein, fusion protein (e.g., PTPR-Fc), peptidomimetic, or modified natural ligand. For example, a PTPR ligand mimetic may bind the same amino acids or a subset of the same amino acids on the PTPR that a natural ligand of the PTPR binds during the physiological functioning of the PTPR. PTPR ligand mimetics include biopolymers (e.g. proteins, nucleic acids, or sugars), lipids, chemical molecules with molecular weights less than five hundred (500) Daltons, one thousand (1000) Daltons, five thousand (5000) Daltons, less than ten thousand (10,000) Daltons, less than twenty five thousand (25,000) Daltons, less than fifty thousand (50,000) Daltons, less than seventy five thousand (75,000), less than one hundred thousand (100,000), or less than two hundred fifty thousand (250,000) Daltons. In embodiments, the synthetic chemical compound is greater than two hundred fifty thousand (250,000) Daltons. In certain embodiments, the PTPR binding agent is less than five hundred (500) Daltons. In embodiments, a PTPR ligand mimetic is a protein. A “PTPRK ligand mimetic” is a PTPRK binding agent that is designed to mimic, in structure or in binding mode, a known RPTPk ligand or is capable of inhibiting the binding of a physiological ligand to RPTPk. In embodiments, the PTPR ligand mimetic is a PTPRK ligand mimetic and inhibits the enzymatic activity of PTPRK. In embodiments, the PTPR ligand mimetic is a PTPRK ligand mimetic which binds at an allosteric site of PTPRK and inhibits the enzymatic activity of PTPRK.

In embodiments, a PTPR ligand mimetic is a small chemical molecule. The term “small chemical molecule” and the like, as used herein, refers to a molecule that has a molecular weight of less than two thousand (2000) Daltons. In embodiments, a small chemical molecule is a molecule that has a molecular weight of less than one thousand (1000) Daltons. In other embodiments, a small chemical molecule is a molecule that has a molecular weight of less than five hundred (500) Daltons. In other embodiments, a small chemical molecule is a molecule that has a molecular weight of less than five hundred (500) Daltons. In other embodiments, a small chemical molecule is a molecule that has a molecular weight of less than one hundred (100) Daltons. In embodiments, the PTPR ligand mimetic is a small chemical molecule PTPRK ligand mimetic and inhibits the enzymatic activity of PTPRK. In embodiments, the PTPR ligand mimetic is a small chemical molecule PTPRK ligand mimetic which binds at an allosteric site of PTPRK and inhibits the enzymatic activity of PTPRK.

An agent may “target” an RPTP, a nucleic acid (e.g. RNA or DNA) of an RPTP, or a protein of an RPTP, by binding (e.g. preferentially binding) to the RPTP, nucleic acid (e.g. RNA or DNA) of an RPTP, or protein of an RPTP. Where preferentially binding, the agent binds preferentially compared to its binding to other molecules of a similar form (e.g. other RPTPs if the agent targets an RPTP). An agent preferentially binds to a molecule, for example, when the binding to the targeted molecule is greater than the binding to other molecules of a similar form. In embodiments, the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In embodiments, an agent targets an RPTP, a nucleic acid (e.g. RNA or DNA) of an RPTP, or a protein of an RPTP, when a binding assay or experiment (e.g. gel electrophoresis, chromatography, immunoassay, radioactive or non-radioactive labeling, immunoprecipitation, activity assay, etc.) reveals only an interaction or primarily an interaction with a single RPTP, a nucleic acid (e.g. RNA or DNA) of a single RPTP, or a protein of a single RPTP. An agent may also “target” an RPTP, a nucleic acid (e.g. RNA or DNA) of an RPTP, or a protein of an RPTP by binding to the RPTP, nucleic acid (e.g. RNA or DNA) of an RPTP, or protein of an RPTP, by decreasing or increasing the amount of RPTP in a cell or organism relative to the absence of the agent, or decreasing the interaction between the RPTP with a physiological or natural ligand. A person having ordinary skill in the art, using the guidance provided herein, may easily determine whether an agent decreases or increases the amount of an RPTP in a cell or organism.

### II. Methods

In a first aspect, there is provided a method of treating an autoimmune disease in a subject in need thereof, the method including administering to the subject an effective amount of a PTPRK antagonist.

In embodiments, the autoimmune disease is a fibroblast mediated disease, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

In embodiments, the autoimmune disease is arthritis. In embodiments, the autoimmune disease is rheumatoid arthritis. In embodiments, the autoimmune disease is psoriatic arthritis.

In embodiments, the disease is non-autoimmune arthritis. In embodiments, the non-autoimmune arthritis is osteoarthritis.

In embodiments, the autoimmune disease is a fibroblast mediated disease. The term “fibroblast mediated disease” and the like refer, in the usual and customary sense, to a disease or disorder involving the presence or action of a fibroblast, either directly or via secretions from the fibroblast, as known in the art. In embodiments, the fibroblast mediated disease is idiopathic pulmonary fibrosis, fibrotic lung diseases, scleroderma, liver fibrosis, liver sclerosis, advanced glomerulonephritis, or nephrosclerosis.

In another aspect, there is provided a method of decreasing inflammation in a synovium of a subject in need thereof, the method including administering to the subject an effective amount of a PTPRK antagonist.

Further to any aspect or embodiment of a method for treating an autoimmune disease or a method for decreasing inflammation in a synovium, in embodiments the subject presents with fibroblast-like synoviocytes that express high levels of PTPRK relative to a standard control as disclosed herein. In embodiments, the subject has rheumatoid arthritis. In embodiments, the standard control is obtained from a disease free subject. In embodiments, the standard control is obtained from a subject not having rheumatoid arthritis.

In another aspect, there is provided a method of decreasing expression of PTPRK in a fibroblast-like synoviocyte, the method including contacting the fibroblast-like synoviocyte (FLS) with an effective amount of a PTPRK antagonist.

In embodiments, the method includes decreasing TNF activity, PDGF activity or IL-1 activity. In embodiments, the method includes decreasing TNF activity. In embodiments, the method includes decreasing PDGF activity. In embodiments, the method including decreasing IL-1 activity.

In embodiments, the method includes decreasing expression of TNF activity, PDGF activity or IL-1 activity. In embodiments, the method includes decreasing expression of TNF activity. In embodiments, the method includes decreasing expression of PDGF activity. In embodiments, the method including decreasing expression of IL-1 activity.

In another aspect, there is provided a method of decreasing invasiveness or migration of a fibroblast-like synoviocyte, the method including contacting the fibroblast-like synoviocyte with an effective amount of a PTPRK antagonist.

Further to any aspect or embodiment of a method for decreasing expression of PTPRK in a fibroblast-like synoviocyte, decreasing TNF activity, IL-1 activity or PDGF activity in a fibroblast-like synoviocyte, or decreasing expression of TNF or ILL In embodiments, the fibroblast-like synoviocyte is a rheumatoid arthritis fibroblast-like synoviocyte. The term “rheumatoid arthritis fibroblast-like synoviocyte” refers to an FLS constituted within or obtained from a subject having rheumatoid arthritis or an FLS that causes, extends or exacerbates RA or symptoms thereof. In embodiments, the fibroblast-like synoviocyte expresses high levels of PTPRK relative to a standard control (e.g., a non-rheumatoid arthritis fibroblast-like synoviocyte).

Further to any aspect or embodiment disclosed above, in embodiments the PTPRK antagonist is an anti-PTPRK antibody, an anti-PTPRK inhibitory nucleic acid or a PTPRK ligand mimetic.

In embodiments, the anti-PTPRK antibody is an anti-PTPRK extracellular antibody. The term “extracellular antibody” in this context refers to an antibody which is directed to an extracellular portion of a target molecule. For example, RPTPκ is expressed as a transmembrane precursor protein that undergoes proteolytic cleavage to generate two non-covalently attached subunits, an N-terminal extracellular subunit, and a C-terminal subunit containing the intracellular and transmembrane regions and a small extracellular region. Thus, an anti-PTPRK extracellular antibody is directed to the extracellular portion of RPTPk.

In embodiments, the anti-PTPRK antibody is an anti-PTPRK dimer inhibiting antibody or an anti-PTPRK dimerizing antibody. The term “dimer inhibiting antibody” refers, in the usual and customary sense, to an antibody which binds a target thereby inhibiting dimerization of the target to form a dimer of target molecules. The term “dimerizing antibody” refers, in the usual and customary sense, to an antibody (e.g., a multivalent antibody, e.g., a divalent antibody) which can bind a plurality (e.g., two) target molecules, thereby forming a dimer of target molecules. In embodiments, the anti-PTPRK antibody is an anti-PTPRK dimer inhibiting antibody. In embodiments, the anti-PTPRK antibody is an anti-PTPRK dimerizing antibody.

In embodiments, the anti-PTPRK inhibitory nucleic acid has at least 50% sequence identity (e.g., 50%, 55%, 60%, 65%, 70%, 75%, 80%, 85%, 90%, 95%, 96%, 97%, 98%, 99% or even 100%) to a contiguous sequence of SEQ ID NO:1 or SEQ ID NO:2 spanning at least 10 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or even greater).

In embodiments, the anti-PTPRK inhibitory nucleic acid has at least 60% sequence identity (e.g., 60%, 65%, 70%, 75%, 80%, 85%, 90%, 95%, 96%, 97%, 98%, 99% or even 100%) to a contiguous sequence of SEQ ID NO:1 or SEQ ID NO:2 spanning at least 10 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or even greater).

In embodiments, the anti-PTPRK inhibitory nucleic acid has at least 70% sequence identity (e.g., 70%, 75%, 80%, 85%, 90%, 95%, 96%, 97%, 98%, 99% or even 100%) to a contiguous sequence of SEQ ID NO:1 or SEQ ID NO:2 spanning at least 10 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or even greater).

In embodiments, the anti-PTPRK inhibitory nucleic acid has at least 80% sequence identity (e.g., 80%, 85%, 90%, 95%, 96%, 97%, 98%, 99% or even 100%) to a contiguous sequence of SEQ ID NO:1 or SEQ ID NO:2 spanning at least 10 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or even greater).

In embodiments, the anti-PTPRK inhibitory nucleic acid has at least 90% sequence identity (e.g., 90%, 91%, 92%, 93%, 94%, 96%, 97%, 98%, 99% or even 100%) to a contiguous sequence of SEQ ID NO:1 or SEQ ID NO:2 spanning at least 10 nucleotides (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or even greater).

In embodiments, the anti-PTPRK inhibitory nucleic acid has at least 50% sequence identity to a contiguous sequence of SEQ ID NO:1 or SEQ ID NO:2 spanning at least 10 nucleotides.

In embodiments, the anti-PTPRK inhibitory nucleic acid has at least 60% sequence identity to a contiguous sequence of SEQ ID NO:1 or SEQ ID NO:2 spanning at least 10 nucleotides.

In embodiments, the anti-PTPRK inhibitory nucleic acid has at least 70% sequence identity to a contiguous sequence of SEQ ID NO:1 or SEQ ID NO:2 spanning at least 10 nucleotides.

In embodiments, the anti-PTPRK inhibitory nucleic acid has at least 80% sequence identity to a contiguous sequence of SEQ ID NO:1 or SEQ ID NO:2 spanning at least 10 nucleotides.

In embodiments, the anti-PTPRK inhibitory nucleic acid has at least 90% sequence identity to a contiguous sequence of SEQ ID NO:1 or SEQ ID NO:2 spanning at least 10 nucleotides.

In embodiments, the anti-PTPRK inhibitory nucleic acid has at least 50% sequence identity to a contiguous sequence of SEQ ID NO:1 or SEQ ID NO:2 spanning at least 15 nucleotides.

In embodiments, the anti-PTPRK inhibitory nucleic acid has at least 60% sequence identity to a contiguous sequence of SEQ ID NO:1 or SEQ ID NO:2 spanning at least 15 nucleotides.

In embodiments, the anti-PTPRK inhibitory nucleic acid has at least 70% sequence identity to a contiguous sequence of SEQ ID NO:1 or SEQ ID NO:2 spanning at least 15 nucleotides.

In embodiments, the anti-PTPRK inhibitory nucleic acid has at least 80% sequence identity to a contiguous sequence of SEQ ID NO:1 or SEQ ID NO:2 spanning at least 15 nucleotides.

In embodiments, the anti-PTPRK inhibitory nucleic acid has at least 90% sequence identity to a contiguous sequence of SEQ ID NO:1 or SEQ ID NO:2 spanning at least 15 nucleotides.

In embodiments, the anti-PTPRK inhibitory nucleic acid has at least 50% sequence identity to a contiguous sequence of SEQ ID NO:1 or SEQ ID NO:2 spanning at least 20 nucleotides.

In embodiments, the anti-PTPRK inhibitory nucleic acid has at least 60% sequence identity to a contiguous sequence of SEQ ID NO:1 or SEQ ID NO:2 spanning at least 20 nucleotides.

In embodiments, the anti-PTPRK inhibitory nucleic acid has at least 70% sequence identity to a contiguous sequence of SEQ ID NO:1 or SEQ ID NO:2 spanning at least 20 nucleotides.

In embodiments, the anti-PTPRK inhibitory nucleic acid has at least 80% sequence identity to a contiguous sequence of SEQ ID NO:1 or SEQ ID NO:2 spanning at least 20 nucleotides.

In embodiments, the anti-PTPRK inhibitory nucleic acid has at least 90% sequence identity to a contiguous sequence of SEQ ID NO:1 or SEQ ID NO:2 spanning at least 20 nucleotides.

In embodiments, the anti-PTPRK inhibitory nucleic acid has at least 50% sequence identity to a contiguous sequence of SEQ ID NO:1 or SEQ ID NO:2 spanning at least 25 nucleotides.

In embodiments, the anti-PTPRK inhibitory nucleic acid has at least 60% sequence identity to a contiguous sequence of SEQ ID NO:1 or SEQ ID NO:2 spanning at least 25 nucleotides.

In embodiments, the anti-PTPRK inhibitory nucleic acid has at least 70% sequence identity to a contiguous sequence of SEQ ID NO:1 or SEQ ID NO:2 spanning at least 25 nucleotides.

In embodiments, the anti-PTPRK inhibitory nucleic acid has at least 80% sequence identity to a contiguous sequence of SEQ ID NO:1 or SEQ ID NO:2 spanning at least 25 nucleotides.

In embodiments, the anti-PTPRK inhibitory nucleic acid has at least 90% sequence identity to a contiguous sequence of SEQ ID NO:1 or SEQ ID NO:2 spanning at least 25 nucleotides.

In embodiments, the anti-PTPRK inhibitory nucleic acid has at least 50% sequence identity to a contiguous sequence of SEQ ID NO:1 or SEQ ID NO:2 spanning at least 30 nucleotides.

In embodiments, the anti-PTPRK inhibitory nucleic acid has at least 60% sequence identity to a contiguous sequence of SEQ ID NO:1 or SEQ ID NO:2 spanning at least 30 nucleotides.

In embodiments, the anti-PTPRK inhibitory nucleic acid has at least 70% sequence identity to a contiguous sequence of SEQ ID NO:1 or SEQ ID NO:2 spanning at least 30 nucleotides.

In embodiments, the anti-PTPRK inhibitory nucleic acid has at least 80% sequence identity to a contiguous sequence of SEQ ID NO:1 or SEQ ID NO:2 spanning at least 30 nucleotides.

In embodiments, the anti-PTPRK inhibitory nucleic acid has at least 90% sequence identity to a contiguous sequence of SEQ ID NO:1 or SEQ ID NO:2 spanning at least 30 nucleotides.

In embodiments, the PTPRK antagonist is an anti-PTPRK inhibitory nucleic acid, wherein the anti-PTPRK inhibitory nucleic acid has at least 90% sequence identity to an at least 10 nucleotide contiguous sequence of SEQ ID NO: 1, SEQ ID NO:2 or a complementary sequence thereof

In embodiments, the PTPRK antagonist is a PTPRK ligand mimetic, wherein the anti-PTPRK ligand mimetic is a peptide or a small chemical molecule.

### III. Pharmaceutical Compositions

In another aspect, there is provided a pharmaceutical composition including a PTPRK antagonist and a pharmaceutically acceptable excipient.

In embodiments, the pharmaceutical composition is for treating an individual who has a disease by administering to the individual a pharmaceutical composition including a therapeutically effective amount of a PTPRK antagonist and a pharmaceutically acceptable excipient. In embodiments, the pharmaceutical composition is for treating an individual who may be at risk of developing a disease by administering to the individual a pharmaceutical composition including a therapeutically effective amount of a PTPRK antagonist and a pharmaceutically acceptable excipient. In embodiments, the disease is an autoimmune disease or disorder, cancer, an infectious disease (e.g. viral, bacterial, parasitic, etc.), an obesity associated disease, a metabolic disease or disorder, an inflammatory disease, an immune disease or disorder, or a traumatic injury. In embodiments, the disease is an inflammatory autoimmune disease (IAD). In embodiments, the disease is a disease associated with a patient's joints. In a certain embodiment, the inflammatory autoimmune disease is rheumatoid arthritis. In embodiments, increased expression of one or more RPTPs is associated with a disease or a risk of developing the disease. In embodiments, decreased expression of one or more RPTPs is associated with a disease or a risk of developing the disease. In embodiments, the increased expression of a first RPTP and the decreased expression of a second RPTP is associated with a disease or a risk of developing the disease.

The PTPRK antagonist may be an anti-PTPRK antibody. In embodiments, the PTPRK antagonist is an anti-PTPR inhibitory nucleic acid. In embodiments, the anti-PTPR inhibitory nucleic acid is an anti-PTPR RNAi molecule. In embodiments, the anti-PTPR inhibitory nucleic acid is an antisense nucleic acid such as anti-PTPR antisense nucleic acid. In embodiments, the PTPRK antagonist is a PTPR ligand mimetic. In embodiments, the PTPR ligand mimetic is a peptide or a small chemical molecule. In embodiments, the PTPR ligand mimetic is an allosteric inhibitor. In embodiments, the PTPRK antagonist is an anti-PTPRK antisense nucleic acid. In embodiments, the PTPRK antagonist is anti-PTPRK antisense nucleic acid.

In embodiments, the pharmaceutical composition is useful for treating an individual who has or may be at risk of developing an inflammatory autoimmune disease. In embodiments, the pharmaceutical compositions are useful for treating an individual who has an inflammatory autoimmune disease by administering to the individual a pharmaceutical composition including a therapeutically effective amount of a PTPRK antagonist and a pharmaceutically acceptable excipient. In embodiments, the pharmaceutical compositions are for treating an individual who may be at risk of developing an autoimmune disease by administering to the individual a pharmaceutical composition including a therapeutically effective amount of a PTPRK antagonist and a pharmaceutically acceptable excipient. In embodiments, the inflammatory autoimmune disease is an arthritis. In embodiments, the autoimmune disease is fibroblast mediated disease, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma. In embodiments, the autoimmune disease is rheumatoid arthritis.

The compositions disclosed herein can be administered by any means known in the art. For example, compositions may include administration to a subject intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a creme, or in a lipid composition. Administration can be local, e.g., to the joint or systemic.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. The percentage of the compositions and preparations may, of course, be varied and may conveniently be between about 2 to about 75% of the weight of the unit, or preferably between 25-60%. The amount of active compounds in such compositions is such that a suitable dosage can be obtained

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

There are provided methods of treating, preventing, and/or ameliorating an autoimmune disorder in a subject in need thereof, optionally based on the diagnostic and predictive methods described herein. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

Administration of a composition for ameliorating the autoimmune disease can be a systemic or localized administration. For example, treating a subject having an autoimmune disorder can include administering an oral or injectable form of PTPRK antagonist on a daily basis or otherwise regular schedule. In embodiments, the treatment is only on an as-needed basis, e.g., upon appearance of autoimmune disease symptoms.

In embodiments, the PTPRK antagonist is an anti-PTPRK antibody, an anti-PTPRK inhibitory nucleic acid or a PTPRK ligand mimetic. In embodiments, the PTPRK antagonist is an anti-PTPRK antibody. In embodiments, the PTPRK antagonist is an anti-PTPRK inhibitory nucleic acid. In embodiments, the PTPRK antagonist is a PTPRK ligand mimetic.

In embodiments, the PTPRK antagonist is an anti-PTPRK extracellular antibody.

In embodiments, the anti-PTPRK antibody is an anti-PTPRK dimer inhibiting antibody or an anti-PTPRK dimerizing antibody.

In embodiments, the PTPRK antagonist is an anti-PTPRK inhibitory nucleic acid, wherein the anti-PTPRK inhibitory nucleic acid as set forth above, including all embodiments thereof.

In embodiments, the PTPRK antagonist is a PTPRK ligand mimetic, wherein the anti-PTPRK ligand mimetic is a peptide or a small chemical molecule.

Any appropriate element disclosed in one aspect or embodiment of a method or composition disclosed herein is equally applicable to any other aspect or embodiment of a method or composition. For example, the therapeutic agents set forth in the description of the pharmaceutical compositions provided herein are equally applicable to the methods of treatment and vice versa.

### IV. Examples

Experimental Methods

Statistics. Two-tailed statistical analyses were performed using GraphPad Prism software (GraphPad Software, La Jolla, Calif.). Unless indicated otherwise, a comparison was considered significant if p was less than 0.05.

Preparation of FLS. FLS lines were obtained from the UCSD Clinical and Translational Research Institute (CTRI) Biorepository. Each FLS line used in this study had been previously obtained from a different patient with either RA or OA. Discarded synovial tissue from patients with OA and RA had been obtained at the time of total joint replacement or synovectomy, as previously described. See e.g., Alvaro-Gracia, J. M. et al., 1991. Journal of immunology 146:3365-3371. The diagnosis of RA conformed to American College of Rheumatology 1987 revised criteria. See e.g., Arnett, F. C. et al., 1988. Arthritis and rheumatism 31:315-324. FLS were cultured in DMEM (Mediatech, Manassas, Va.) with 10% fetal bovine serum (FBS, Omega Scientific, Tarzana, Calif.), 2 mM L-glutamine, 50 μg/mL gentamicin, 100 units/ml of penicillin and 100 μg/ml streptomycin (Life Technologies, Carlsbad, Calif.) at 37° C. in a humidified 5% CO₂ atmosphere. Cells in this study were synchronized in 0.1% FBS (serum-starvation media) for 48 hr prior to analysis or functional assays.

Antibodies and Other Reagents. The rabbit anti-RPTPκ antibody was a kind gift from Axel Ullrich (Max Planck Institute of Biochemistry). The anti-cadherin-11 antibody was purchased from Life Technologies. All other primary antibodies were purchased from Cell Signaling Technology (Danvers, Mass.). Secondary antibodies were purchased from GE Healthcare Life Sciences (Pittsburgh, Pa.). TGFβ1, TNFα, IL-1β and PDGF-BB were purchased from eBioscience (San Diego, Calif.). Control non-targeting, anti-PTPRK and anti-PTPRM antisense oligonucleotides (ASO) were purchased from Gene Tools, LLC (Philomath, Oreg.). Chemical inhibitors PP2 and U73122 were purchased from EMD Millipore (Billerca, Mass.). Horseradish peroxidase (HRP)-conjugated S protein was purchased from EMD Millipore. Unless otherwise specified, chemicals and all other reagents were purchased from Sigma-Aldrich.

Quantitative Real-Time RT-PCR (qPCR). PTPRK expression can be assessed by qPCR normalized, e.g., to the housekeeping gene RPII, and plotted relative to the PTPRK expression in a control, e.g., Ctl ASO-treated cells. Following treatment of RA FLS for a given time, e.g., 7 days, with 2.5 μM Ctl or PTPRK ASO, TGFB1 expression can be assessed by qPCR, normalized to the housekeeping gene RPII, and plotted relative to the TGFB1 expression in Ctl ASO-treated cells.

Following cell synchronization for 48 hr, cells were stimulated as indicated for 24 hr or left unstimulated. RNA was extracted using RNeasy® Kits (Qiagen, Valencia, Calif.). cDNA was synthesized using the SuperScript® III First-Strand Synthesis SuperMix for qRT-PCR (Life Technologies, Carlsbad, Calif.). qPCR was performed using a Roche Lightcycler® 480 (Indianapolis, Ind.), with individual primer assays and SYBR® Green qPCR Mastermix purchased from SABiosciences/Qiagen. Efficiency of the primer assays was guaranteed by the manufacturer to be greater than 90%. Each reaction was measured in triplicate and data was normalized to the expression levels of the house-keeping gene RNA Polymerase II (RPII) (Radonic, A. et al., 2004. Biochemical and biophysical research communications 313:856-862), also measured in triplicate. Absence of genomic DNA contamination was confirmed using control reactions lacking the reverse transcriptase enzyme during the cDNA synthesis step.

Sequences of ASOs disclosed herein include:

| (SEQ ID NO: 3) | |
|---|---|
| PTPRK ASO: | TCTTAATCACAACCTACCACAAGGA; |
| (SEQ ID NO: 4) | |
| PTPRK_2 ASO: | ACAGCAAAGTATGAGCATACCATCT; |
| (SEQ ID NO: 5) | |
| PTPRM ASO: | TAAAGACAACTTACTACATGGATGT; |
| (SEQ ID NO: 6) | |
| Control ASO: | CCTCTTACCTCAGTTACAATTTATA |

Cell Lysis for Western Blotting and RPTPκ Immunoprecipitation. RPTPκ can be immunoprecipitated from RA FLS using an anti-RPTPκ antibody that recognizes the juxtamembrane region of the protein. Both the full-length precursor (210 kDa) and membrane-bound intracellular furin-cleavage product (110 kDa) can be detectable. RPTPκ is expressed in RA synovial lining. The expression of PTPRK and a positive control (e.g., IL6) can be measured by qPCR in RA and OA FLS lines following cell stimulation with e.g., 50 ng/ml TNF or 2 ng/ml interleukin 1 (IL-1β) for 24 hr.

Cells were lysed in RIPA buffer (25 mM Tris-HCl pH 7.6, 150 mM NaCl, 1% NP-40, 1% sodium deoxycholate, 0.1% SDS) containing 1 mM phenylmethanesulfonyl fluoride, 10 μg/ml aprotinin, 10 μg/ml leupeptin, 10 μg/ml soybean trypsin inhibitor, 1-10 mM sodium orthovanadate, 5 mM sodium fluoride and 2 mM sodium pyrophosphate. Protein concentration of cell lysates was determined using the Pierce BCA Protein Assay Kit (Thermo Scientific, Rockford, Ill.).

Immunohistochemistry of Synovial Tissue. Paraffin embedded slides of human RA synovial tissue were obtained from the UCSD CTRI Biorepository. Slides were pretreated for 10 min with boiling citrate antigen retrieval buffer (1.9 mM citric acid, 10 mM Tris-sodium citrate pH 6.0) before being treated with 3% H₂O₂ for 10 min. Slides were then blocked with 5% goat serum for 1 hr at room temperature. Rabbit anti-RPTPκ antibody or goat anti-rabbit IgG (1:100 in 5% bovine serum albumin [BSA]) were incubated with the slides overnight at 4° C. The slides were then washed and incubated with peroxidase-linked goat anti-rabbit IgG secondary antibody (Life Technologies, 1:300 in 5% BSA) for 1 hr, and then incubated for 5 min with 3,3′-diaminobenzidine substrate (Sigma-Aldrich). Slide images were obtained using an Eclipse 80i microscope (Nikon, Melville, N.Y.).

FLS Treatment with ASO. Cells were treated with 2.5 μM ASO for a total of 7 days. ASO was replaced in fresh culture medium after 3 d and replaced in cell synchronization medium after 5 days. After ASO treatment, cells can be stimulated with additional agent, e.g., 50 ng/ml TGFβ1, or left unstimulated, in the presence of ASO for 24 hr.

FLS Transwell Invasion Assays. The in vitro invasion assays were performed in transwell systems as previously described (Laragione, T. et al., 2008. Arthritis and rheumatism 58:2296-2306; Tolboom, T. C. et al., 2005. Arthritis and rheumatism 52:1999-2002). RA FLS were pre-treated with ASO for 5 days, and synchronized in the presence of ASO for an additional 2 days, and then subjected to the invasion assays. FLS (2.5-5×10⁵) were resuspended in assay media (DMEM with 0.5% BSA) and allowed to invade through BD BioCoat™ GFR Matrigel™ Invasion Chambers (BD Biosciences) in response to 50 ng/ml platelet-derived growth factor BB (PDGF-BB) for 24 hr. For visualization, cells were either pre-stained with 2 μM CellTracker Green™ (Life Technologies) or stained post-invasion with 2 μM Hoechst (Life Technologies) for 30 min at room temperature. Fluorescence of invading cells on each membrane was visualized using an Eclipse 80i microscope. Images were acquired from 4 non-overlapping fields per membrane, and invading cells in each field were counted using ImageJ software. Each experiment included 3-4 membranes per sample.

FLS Transwell Migration Assays. The transwell migration assays were performed similarly to the invasion assays. For experiments with ASO, RA FLS were pre-treated with ASO for 5 days, and synchronized in the presence of ASO for an additional 2 days. For experiments with chemical inhibitors, cells were synchronized for 48 hr and then pre-treated with compound or dimethylsulfoxide (DMSO) for 30 min. FLS were allowed to migrate through uncoated transwell chambers in response to 5% FBS for the times indicated in the figure legend. For visualization, cells were either pre-stained with 2 μM CellTracker Green™ or stained post-invasion with 2 μM Hoechst for 30 in at room temperature. Fluorescence of migrating cells on each membrane was visualized as above. Each experiment included 3-4 membranes per sample.

FLS Modified Spheroid Migration Assay. The modified spheroid migration assays were performed as in (Bartok, B. et al., 2014. J Immunol 192:2063-2070). RA FLS were pre-treated with ASO for 5 days, and synchronized in the presence of ASO for an additional 2 days. Cells were resuspended at a concentration of 1×10⁶ cells/50 μl in DMEM. Cells (2 μl) were mixed at 1:1 in 4 mg/ml growth factor-reduced BD Matrigel Matrix (BD Biosciences), pipetted as a spot in a 24-well tissue culture dish, and incubated at 37° C. for 15 min to gel. Serum-starvation medium with or without PDGF-BB (10 ng/ml) was added. Cell movement concentrically was monitored after 2 d. At the end of the experiment, cells were fixed and stained using the Hemacolor staining kit (EMD Millipore). Images were acquired from 4 fields per spot, and migrated cells were counted using ImageJ software.

FLS Spreading and Adhesion Assays. RA FLS were pre-treated with ASO for 5 days, and synchronized in the presence of ASO for an additional 2 days. Equal cell numbers were then resuspended in FLS medium containing 5% FBS and allowed to adhere onto circular coverslips coated with 20 μg/ml fibronectin (FN) at 37° C. for 15 min (adhesion assays) or 15, 30 and 60 min (spreading assays). Following the incubation period, cells were fixed in 4% para-formaldehyde for 5 min, permeabilized in 0.2% Triton X-100 for 2 min, and stained with 5 U/ml Alexa Fluor® 568 (AF 568)-conjugated phalloidin and 2 μg/ml Hoechst for 20 min each (Life Technologies). Samples were imaged with an Olympus FV10i Laser Scanning Confocal microscope (Olympus, Center Valley, Pa.). Using the FV10i acquisition software, each circular coverslip of cells was separated into four nine-paneled mega-images. Each panel (1024×1024) was optically acquired with a 10× objective using the FV10i acquisition software and stitched together, through a 10% overlap, with the Olympus FluoView™ 1000 imaging software. Total cell number and cell areas for each panel were calculated using Image Pro Analyzer software (Media Cybernetics, Rockville, Md.).

FLS Survival and Apoptosis Assay. RA FLS were pre-treated with ASO for 5 days, and synchronized in the presence of ASO for an additional 2 days. Cells were washed and incubated for an additional 24 h in serum-starvation media. Adherent and non-adherent cells were collected and stained with Annexin V-Alexa Fluor® 647 (AF 647) and propidium iodide according to the manufacturer's instructions (Biolegend, San Diego, Calif.). Cell fluorescence was assessed by FACS using a BD LSR-II (BD Biosciences).

Synovial Micromass Organ Cultures. ASOs disclosed herein can enable efficient knockdown of RPTPκ. For example, RA FLS can be treated with 2.5 μM Ctl or PTPRK_2 ASO for 7 days. PTPRK expression can be assessed by qPCR, normalized to the housekeeping gene RPII, and plotted relative to the PTPRK expression in Ctl ASO-treated cells. Following treatment with 2.5 μM Ctl or PTPRK_2 ASO for 7 d, RA FLS can be assayed for invasion through Matrigel-coated transwell chambers in response to 50 ng/ml PDGF-BB for 24 hr. PTPRM ASO enables efficient knockdown of PTPRM. RA FLS can be treated with 2.5 μM Ctl or PTPRM ASO for 7 days. PTPRM expression can be assessed by qPCR, normalized to the housekeeping gene RPII, and plotted relative to the PTPRM expression in Ctl ASO-treated cells. Panel shows mean±range. Following treatment with 2.5 μM Ctl or PTPRM ASO for 7 d, RA FLS can be assess for invasion through Matrigel-coated transwell chambers in response to 50 ng/ml PDGF-BB for 24 hr. Following treatment with ASO (e.g., Ctl ASO or PTPRK ASO) and cell synchronization, RA FLS can be serum-starved (FLS medium with 0.1% FBS) for an additional 24 hr. Cells can be collected and stained (e.g., with Annexin V and PI), and cell fluorescence can be assessed by FACS.

Accordingly, synovial organ cultures were prepared as described in (Kiener, H. P. et al., 2009. Arthritis and rheumatism 60:1305-1310). RA FLS were suspended in ice-cold Matrigel Matrix (BD Biosciences) at 2×10⁶ cells/ml. Twenty-five-μl droplets of the suspension were placed onto culture dishes coated with poly-2-hydroxyethyl-methacrylate (Aldrich Chemical Co., Milwaukee, Wis.) and allowed to gel for 30 minutes at 37° C. Gels were overlaid with FLS culture medium supplemented with nonessential amino acid solution, ITS (Insulin-Transferrin-Selenium, Life Technologies) and 0.1 mM ascorbic acid. The floating three-dimensional culture was maintained for 3 weeks, with the medium replaced twice per week. After 2 weeks, 2.5 μM Ctl or PTPRK ASO was added to the medium to induce knockdown (KD) of PTPRK expression during the final week of culture. Micromasses were fixed in 10% neutral buffered formalin, embedded in paraffin, sectioned at 6 micron-thick sections, and stained with hematoxylin and eosin.

Beta-Catenin and SMAD3 Subcellular Localization Assays. Following treatment with ASO (e.g., Ctl ASO or PTPRK ASO over time (e.g., 5-7 days), RA FLS can be stimulated with 50 ng/ml TGFβ1 for 5, 15, 30 or 60 min, or left unstimulated. Assay by Western blotting of cell lysates can be conducted with anti-phospho-SMAD3 and anti-SMAD3 antibodies, phalloidin and Hoechst and can be imaged by immunofluorescence microscopy.

Accordingly, RA FLS were plated on glass coverslips. Cells were pre-treated with ASO for 5 days, and synchronized in the presence of ASO for an additional 2 days. Cells were then stimulated with 50 ng/ml TGFβ1 for 30 min or 24 hr, or left unstimulated, and then fixed in 4% para-formaldehyde for 5 min, permeabilized in 0.2% Triton X-100 for 2 min, and stained for 1 hr each with anti-SMAD3 antibody (Cell Signaling Technology) and anti-beta-catenin antibody (BD Biosciences), and AF 488 goat anti-rabbit secondary antibody and AF 647 goat anti-mouse secondary antibody (Life Technologies). Cells were then stained with 5 U/ml AF 568-conjugated phalloidin and 2 μg/ml Hoechst for 20 min each. Samples were imaged with an Olympus FV10i Laser Scanning Confocal microscope (Olympus, Center Valley, Pa.). Using the FV10i acquisition software, each circular coverslip of cells was imaged by using 3 random fields of view and acquiring a stitched nine paneled mega-image. Each panel (1024×1024) was optically acquired with a 60× objective using the FV10i acquisition software and stitched together, through a 10% overlap, with the Olympus FluoView™ 1000 imaging software. Each mega-image was then further processed, post stitching, using Image Pro Analyzer software (Media Cybernetics, MD). Using Image Pro, the original mega images were used to first automatically define nuclear localization by masking the Hoechst nuclear signal then isolating the fluorescence of either SMAD3 or beta-catenin signal localized within the nucleus mask. Cytoplasmic signals were defined by removing the masked nuclear signal from the images and thus quantifying the remaining cytoplasmic signal of SMAD3 or beta-catenin. Total area of nuclear and cytoplasmic fluorescence label of either SMAD3 or beta-catenin were calculated.

COS-1 Transfection. cDNA encoding C-terminally HA-tagged wild type (WT) or catalytically inactive (C1100S) RPTPκ (NM 008983.2) was cloned into the pcDNA3.1(+) vector. COS-1 cells were cultured in DMEM with 10% FBS, 100 units/ml of penicillin and 100 μg/ml streptomycin. Cells were transfected with Lipofectamine 3000 (Life Technologies) according to the manufacturer's protocol, and harvested after 2 d for analysis.

In vitro Pull-Down Assay. HA-tagged WT RPTPκ was overexpressed in COS-1 cells. Cells were lysed in THE buffer (20 mM Tris-HCl pH 7.5, 150 mM NaCl, 5 mM EDTA pH 8.0 and 1% NP-40) containing 10 μg/mL aprotinin, 10 μg/mL leupeptin, 10 μg/mL soybean trypsin inhibitor, and 1 mM phenylmethylsulfonyl fluoride. RPTPκ was immunoprecipitated using anti-HA antibody (Covance, Princeton, N.J.). RA FLS were lysed in RIPA buffer containing 10 μg/mL aprotinin, 10 μg/mL leupeptin, 10 μg/mL soybean trypsin inhibitor, 1 mM phenylmethylsulfonyl fluoride and 5 mM iodoacetamide. Lysates were treated with 10 mM dithiothreitol for 10 min, and then diluted 10-fold in buffer containing 25 mM Tris-HCl pH 7.6, 150 mM NaCl and 1 mM ethylenediaminetetraacetic acid. Immunoprecipitates were incubated with RA FLS lysates for 3 hr at 4° C., washed in the dilution buffer, and subjected to Western blotting.

Substrate-Trapping Pull-down Assay. Substrate-trapping is a well-established technique to identify substrates of PTPs (Garton, A. J. et al., 1996. Mol Cell Biol 16:6408-6418). PTP substrate-trapping involves mutation of a residue, typically an aspartic acid essential for catalysis, in the catalytic domain of the PTP. Substrates can bind the catalytic cleft, but catalysis is not completed, leading to formation of a complex in which the substrate is “trapped” by the PTP. SRC is the substrate of RPTPκ in RA FLS. A substrate-trapping mutant of iPTPκ (D1051A) can trap SRC from RA FLS. Agarose-bound S-tagged-iPTPκ-D1051A can be incubated in vitro with RA FLS lysates, and the pull-down can be subjected to Western blotting and probed using HRP-conjugated S-protein. RA FLS can be stimulated with 100 μM pervanadate for 15 min immediately prior to lysis. RPTPκ dephosphorylates a SRC Y527 phospho-peptide. Immunoprecipitated wild type (WT) or catalytically inactive C1100S (C/S) HA-tagged RPTPκ can be incubated in vitro with SRC pY527 peptide for 30 min. The reaction can be stopped with addition of Biomol Green.

Accordingly, cDNA (codon optimized for expression in E. coli [Genscript, Piscataway, N.J.]) encoding a substrate-trapping mutant of the catalytic domain of RPTPκ (iPTPK-D1051A, aa865-1156 of NP_002835.2) was cloned into the pET30c vector. S-tagged-iPTPK-D1051A was isolated from E. coli using S-protein agarose (EMD Millipore). RA FLS were lysed in RIPA buffer containing 10 μg/mL aprotinin, 10 μg/mL leupeptin, 10 μg/mL soybean trypsin inhibitor, 1 mM phenylmethylsulfonyl fluoride and 5 mM iodoacetamide. Lysates were treated with 10 mM dithiothreitol for 10 min, and then diluted 10-fold in buffer containing 25 mM Tris-HCl pH 7.6, 150 mM NaCl and 1 mM ethylenediaminetetraacetic acid. Agarose-bound S-tagged-iPTPκ-D1051A was incubated with RA FLS lysates for 3 hr at 4° C., washed in the dilution buffer, and subjected to Western blotting.

In vitro PTP Assay. HA-tagged WT and catalytically inactive mutant C1100S (C/S) RPTPκ were overexpressed in COS-1 cells. Cells were lysed in HNE buffer (50 mM Hepes, pH 7.4, 150 mM NaCl, 5 mM EDTA and 1% Triton-X) containing 10 μg/mL aprotinin, 10 μg/mL leupeptin, 10 μg/mL soybean trypsin inhibitor, 1 mM phenylmethylsulfonyl fluoride. RPTPκ was immunoprecipitated using anti-HA antibody. Immunoprecipitates were washed extensively in 50 mM Bis-Tris pH 6.0, and incubated with 50 mM Bis-Tris pH 6.0 and 5 mM DTT for 30 min at 4° C. Immunoprecipitates were divided into triplicate reactions and incubated with 0.2 mM phospho-SRC Y527 peptide [H-TSTEPQ-pY-QPGENL-OH] (Anaspec, Fremont, Calif.) in 50 mM Bis-Tris pH 6.0, 5 mM DTT and 0.005% Tween-20 at 37° C. for 30 min. Reactions were stopped with the addition of Biomol Green (Enzo Life Sciences, Plymouth Meeting, Pa.), and absorbance of the solution was measured at 620 nm using a Tecan M1000 plate-reader (Tecan Systems, San Jose, Calif.). PTP activity was plotted as absorbance following subtraction of absorbance from the blank reactions (control anti-HA immunoprecipitations from lysates of cells transfected with empty vector), also measured in triplicate.

In vivo Invasion Assay. The in vivo invasion assay was performed as described in (You, S. et al., 2014. Proceedings of the National Academy of Sciences of the United States of America 111:550-555) Skin inflammation was induced in athymic nude mice by subcutaneously injecting 120 μg complete Freund's adjuvant (CFA) in each flank. The next day, 5×10⁵ RA FLS pretreated with Ctl or PTPRK ASO were intradermally implanted 1.2 cm distance from the CFA injection sites (each mouse was injected with Ctl ASO-treated cells in one flank and PTPRK ASO-treated cells in the contralateral flank). After 5 days, the skin regions between the 2 injection sites was harvested, and FLS invasion from the implantation site towards the inflammation site was assessed by immunohistochemical staining with an anti-HLA Class I antibody (Abcam, Cambridge, Mass.) Skin samples were frozen in optimal cutting temperature compound (OCT), and 2 cryosections from immediately adjacent the CFA injection site were obtained from each skin sample. Cryosections were fixed in 4% para-formaldehyde for 10 min and pretreated for 10 min with boiling citrate antigen retrieval buffer (1.9 mM citric acid, 10 mM Tris-sodium citrate, pH 6.0) before being treated with 3% H₂O₂ for 10 min. Slides were blocked with 5% BSA overnight at 4° C., then incubated with anti-human HLA Class I antibody (1:200 in 5% BSA) for 1 hr at room temperature. The slides were then washed and incubated with peroxidase-conjugated anti-mouse IgG from Vector Laboratories for 30 min at room temperature, and then incubated for 5 minutes with 3,3′-diaminobenzidine substrate and stained with hematoxylin. Samples were then imaged using a BZ-9000E microscope (Keyence, Itasca, Ill.). The numbers of invaded FLS, recognized by staining with the anti-human HLA Class I antibody, in each 20× field were manually counted.

### Experimental Results and Discussion

### Example 1

### PTPRK Expression is Upregulated in RA FLS

Comparison of PTP expression in FLS from 3 RA and 3 OA patients revealed increased PTPRK mRNA in RA FLS. PTPRK encodes the receptor PTPκ (RPTPκ), a transmembrane PTP reported to regulate cell growth and migration through dephosphorylation of protein tyrosine kinases, cadherin family proteins, and beta-catenin. See e.g., Wang, S. E. et al., 2005. Mol Cell Biol, 25:4703-4715; Novellino, L. et al., 2008. Cell Signal, 20:872-883; Xu, Y. et al., 2009. J Cell Biochem, 107:873-880. RPTPκ is expressed as a transmembrane precursor protein that undergoes furin-mediated proteolytic cleavage to generate two non-covalently attached subunits, an N-terminal extracellular subunit, and a C-terminal subunit containing the intracellular and transmembrane regions and a small extracellular region. See e.g., Jiang, Y. P. et al., 1993. Mol Cell Biol, 13:2942-2951. RPTPκ belongs to a transmembrane PTP subfamily, including RPTPμ(encoded by PTPRM), RPTPρ (encoded by PTPRT) and RPTPψ (encoded by PTPRU), characterized by an extracellular region of a Meprin-A5-protein PTPμ domain, an Immunoglobulin-like domain, and 4 Fibronectin III-type repeats, and an intracellular region containing a juxtamembrane region and two tyrosine PTP domains, of which only the first has catalytic activity. See e.g., Andersen, J. N. et al., 2001. Molecular and cellular biology, 21:7117-7136. Although PTPRM is also highly expressed in FLS (Stanford, S. M. et al., 2013. Arthritis Rheum, 65:1171-1180), we did not detect differential expression of this gene, or any other transmembrane PTP, between RA and OA FLS.

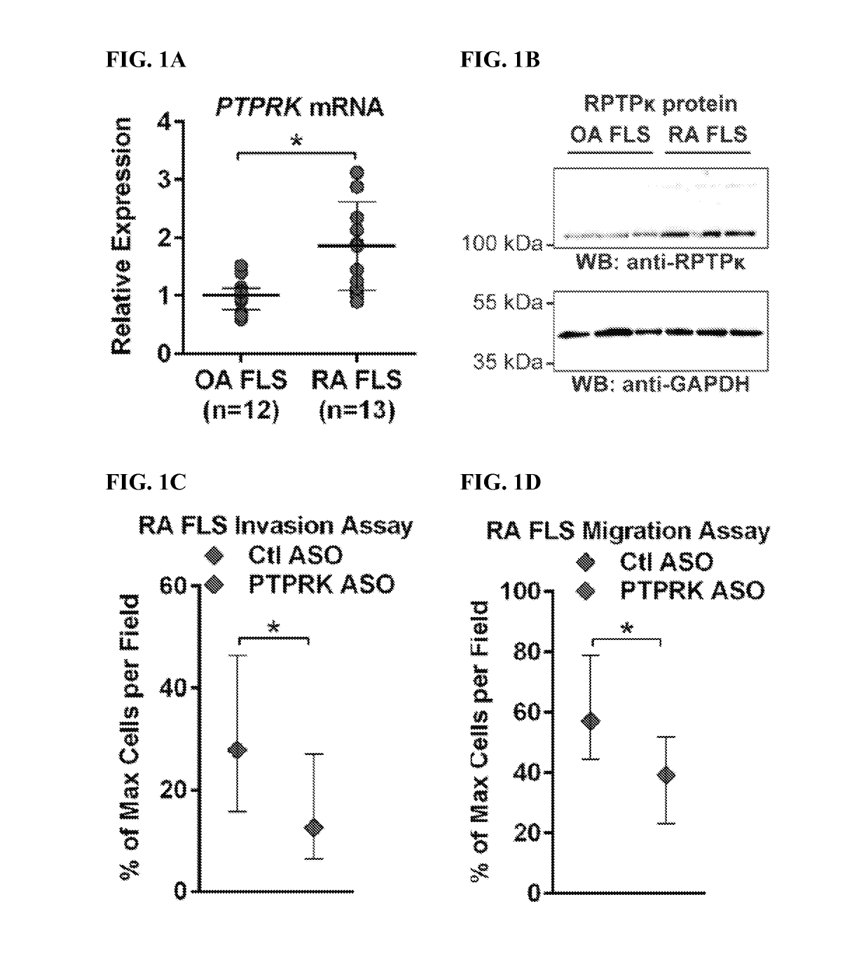

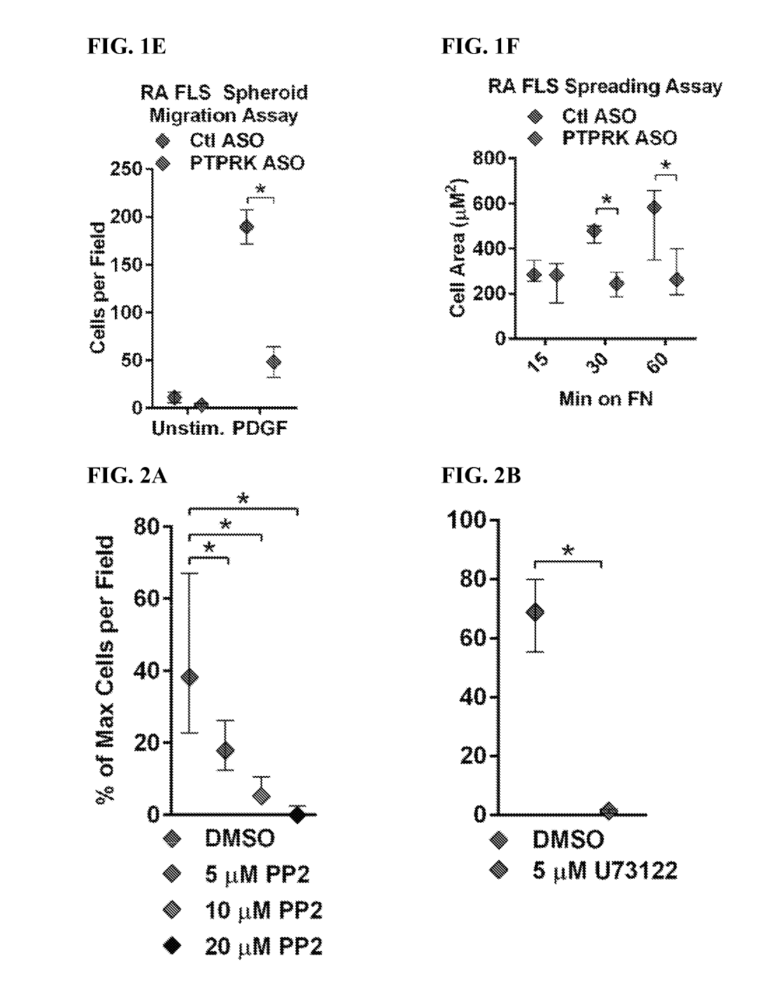

The expression of PTPRK was retested in a further set of FLS lines from 13 RA and 12 OA patients, confirming significantly increased (1.86-fold) PTPRK expression in RA FLS (FIG. 1A). Using an antibody recognizing the RPTPκ intracellular juxtamembrane region, we detected the presence of both the RPTPκ precursor (210 kDa) and C-terminal cleavage product in RA FLS, and importantly, increased expression of RPTPκ protein in RA compared to OA FLS (FIG. 1B).

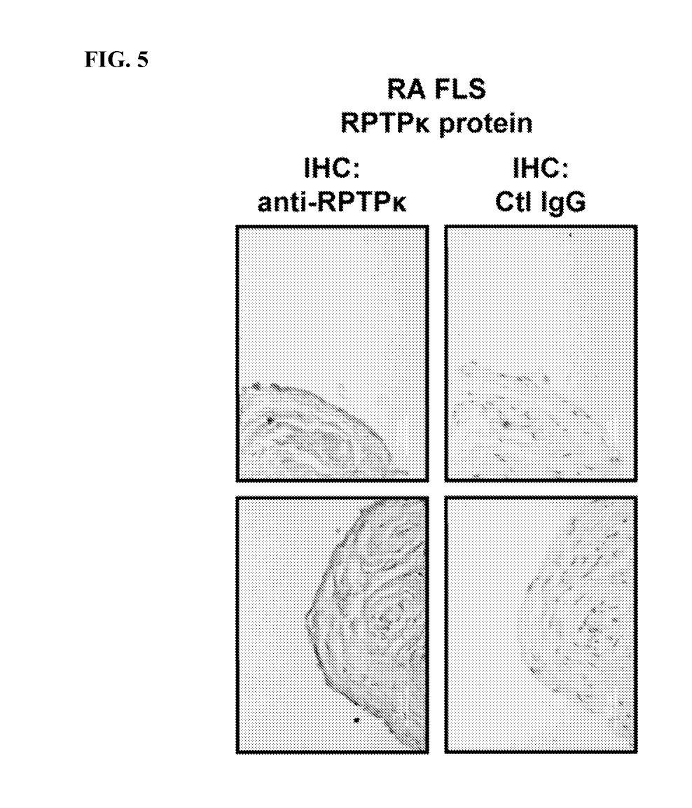

To verify RPTPκ expression in the primary rheumatoid synovial lining, we performed immunohistochemistry on synovial sections obtained from biopsies of RA patients. RPTPκ expression was detected in the rheumatoid synovium, with prominent expression in the synovial intimal lining. See FIG. 5.

### Example 2

### PTPRK Overexpression in RA FLS is TGFB1-Dependent

It has been reported that RA FLS express higher levels of TGFB1 than OA FLS. See e.g., Pohlers, D. et al., 2007. Arthritis Res Ther, 9:R59. As PTPRK is a reported TGFβ/SMAD target gene (Wang, S. E. et al., 2005, Id.), without wishing to be bound by any theory we reasoned that the increased PTPRK expression in RA FLS may be due to increased expression of TGFB1. Accordingly, PTPRK and PTPRM mRNA expression in RA FLS were measured following cell stimulation with 50 ng/ml recombinant TGFβ1 for 24 hr. The expression of PTPRK, and as a positive control IL6, was measured by qPCR in 8 RA and 8 OA FLS lines following cell stimulation with 50 ng/ml TNF or 2 ng/ml interleukin 1 (IL-1β) for 24 hr. PTPRK, but not PTPRM, expression was increased 1.90-fold in response to treatment of cells with TGFβ1 but not in response to the inflammatory cytokines tumor necrosis factor (TNF) or interleukin 1 (IL-1).

We next confirmed the trend of increased expression of TGFB1 in RA FLS. See e.g., Pohlers, D. et al., 2007. Id. PTPRK and TGFB1 mRNA expression in FLS was measured, which provided a significant positive correlation between the expression levels of PTPRK and TGFB1 in the RA FLS (Spearman r=0.5824, p<0.05), but not in the OA FLS.

We then tested if this was due to TGFB1-mediated upregulation of PTPRK, or to PTPRK-mediated potentiation of TGFB1 expression. Cell-permeable antisense oligonucleotide (ASO) enables efficient knockdown of PTPRK expression in RA FLS. RA FLS were treated with 2.5 μM control (Ctl) or PTPRK ASO for 7 days. After 6 days of ASO treatment, cells were stimulated with 50 ng/ml TGFβ1, or left unstimulated, in the presence of ASO for 24 hr. Accordingly, we subjected RA FLS to knockdown of PTPRK expression using cell-permeable antisense oligonucleotide (ASO) targeted against PTPRK (PTPRK ASO), and found TGFB1 expression was unaffected by PTPRK deficiency. RA FLS were treated with 25 μM SB505124 or DMSO for 30 min, then stimulated with 10 ng/ml TGFβ1 or left unstimulated for 24 hr, which showed that, in contrast, treatment of RA FLS with the TGFβ type 1 receptor chemical inhibitor SB505124 reduced the basal and TGFβ-stimulated levels of PTPRK.

### Example 3

### RPTPκ Promotes Invasiveness of RA FLS

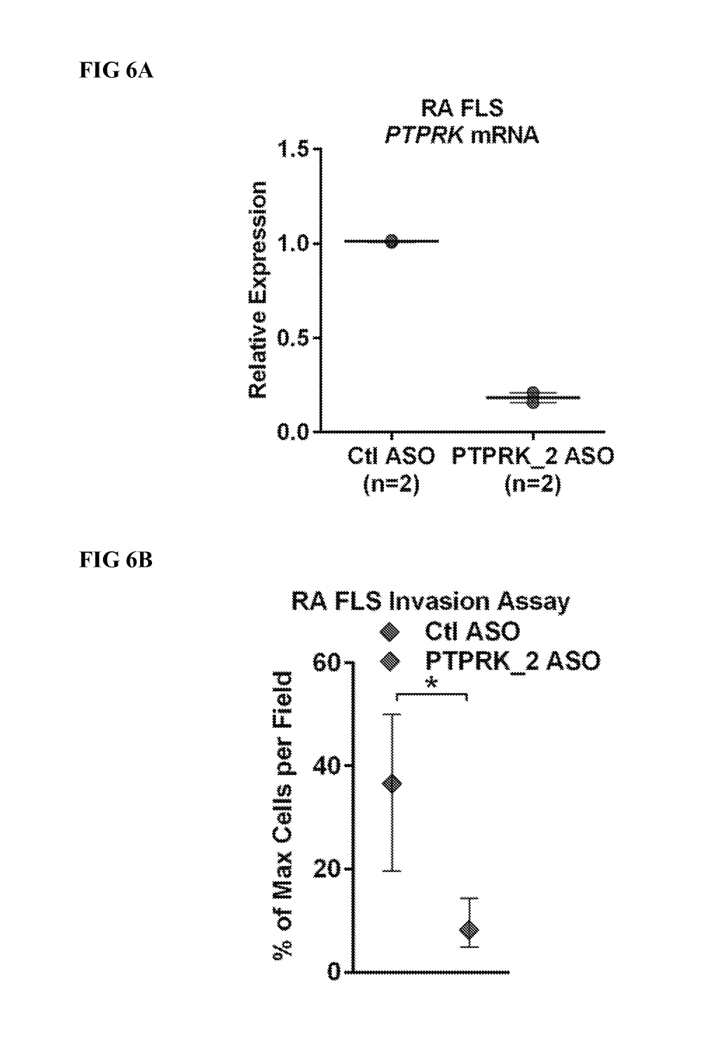

We next tested if reduction in RPTPκ expression could inhibit the ex vivo invasiveness of RA FLS, a phenotype correlated with radiographic damage in RA. See e.g., Tolboom, T. C. et al., 2005, Id. We subjected the ASO-treated RA FLS to transwell invasion assays through Matrigel matrix in response to platelet-derived growth factor (PDGF), a prominent growth factor in the RA synovium that promotes FLS invasiveness. See e.g., Bottini, N., & Firestein, G. S. 2013. Id. Following treatment with control (Ctl) or PTPRK ASO for 7 d, RA FLS invaded through Matrigel-coated transwell chambers in response to 50 ng/ml PDGF-BB for 24 hr. RA FLS treated with PTPRK ASO, compared to control non-targeting ASO-treated cells, were significantly less invasive in response to PDGF (FIG. 1C). The effect was replicated by treatment of RA FLS with a second PTPRK-targeted ASO (PTPRK_2 ASO) (see e.g., FIG. 6A-6B), but not by treatment of cells with a PTPRM-targeted ASO.

We next assessed the effect of PTPRK ASO on RA FLS migration. PTPRK ASO-treated cells showed significantly reduced migration in a transwell assay in response to 5% fetal bovine serum (FBS) (FIG. 1D), and out of a sphere of Matrigel in response to PDGF (FIG. 1E). We hypothesized this effect was due to increased cell death or reduced cytoskeletal reorganization following RPTPκ knockdown. Treatment of RA FLS did not increase cell apoptosis or necrosis, but significantly reduced cell spreading (FIG. 1F).

### Example 4

### RPTPκ Promotes RA FLS Migration Through Dephosphorylation of SRC

We investigated the molecular mechanism by which RPTPκ knockdown impairs invasion and migration of RA FLS in response to PDGF. We found no reduction in expression of the PDGF receptor (PDGFR) upon PTPRK ASO treatment. We explored RPTPκ candidate substrates with a role in cell migration; e.g., RPTPk has been proposed to interact with cadherin and catenin proteins. See e.g., Novellino, L. et al., 2008. Cell Signal, 20:872-883; Lilien, J., & Balsamo, J. 2005. Curr Opin Cell Biol, 17:459-465; Anders, L. et al., 2006. Mol Cell Biol, 26:3917-3934. Cadherin-11 is highly expressed in FLS and is critical for FLS invasiveness and for formation and maintenance of the synovial lining. See e.g., Noss, E. H., & Brenner, M. B., 2008. Immunological reviews, 223:252-270. We detected basal tyrosine phosphorylation of cadherin-11 immunoprecipitated from RA FLS, which was unaffected by RPTPk knockdown. Additionally, RPTPk knockdown had no effect on RA FLS ability to form a synovial lining layer in an in vitro organ culture. Tyrosine phosphorylation of beta-catenin has been proposed to promote cell migration and regulate beta-catenin nuclear recruitment and transcriptional activity. See e.g., Lilien, J., & Balsamo, J. 2005. Curr Opin Cell Biol, 17:459-465. However, we found no increase in beta-catenin tyrosine phosphorylation, nor alterations in the ratio of beta-catenin cytosolic/nuclear localization, upon RPTPκ knockdown (FIG. 4).

In mammary cells, RPTPk promotes receptor tyrosine kinase (RTK) signaling pathways through dephosphorylation of the C-terminal inhibitory tyrosine residue (Y527 of SRC) of SRC family kinases (SFKs). See e.g., Wang, S. E. et al., 2005, Id. It is believed that dephosphorylation of this site enhances SFK activity and promotes signaling of downstream RTKs, including growth factor-induced activation of mediators of cell motility and invasion, phospholipase C-y 1 (PLCyl) and focal adhesion kinase (FAK). See e.g., Mitra, S. K., Hanson, D. A., and Schlaepfer, D. D. 2005. Molecular cell biology, 6:56-68; Yu, H. et al., 1998. Exp Cell Res, 243:113-122. We assessed by Western blotting if RPTPκ promotes RA FLS motility through SRC-dependent PDGFR signaling. Western blotting was conducted on ASO-treated RA FLS lysates, and ASO-treated RA FLS stimulated with 50 ng/ml PDGF-BB for 30 min or left unstimulated. RA FLS treated with PTPRK ASO showed increased basal phosphorylation of SRC Y527, and reduced PDGF-induced phosphorylation of PLCyl (Y783) and FAK (Y925). The essential roles of SFK and PLCyl activity in RA FLS motility were confirmed using pharmacological inhibitors of these enzymes. Treatment with the SFK inhibitor PP2 (Hanke, J. H. et al., 1996. J Biol Chem, 271:695-701) or the PLCyl inhibitor U73122 (Smith, R. J. 1990. J Pharmacol Exp Ther, 253:688-697) (FIGS. 2A-2B) abolished growth factor-induced migration of RA FLS.

We next investigated if RPTPk directly interacts with and/or dephosphorylates SFKs. HA-tagged RPTPκ was immunoprecipitated from COS-1 cells and incubated in vitro with RA FLS lysates, and pull-down was subjected to Western blotting. Both full-length RPTPk immunoprecipitated from COS-1 cells and a recombinant substrate-trapping mutant of the RPTPk catalytic domain (iPTPk-D1051A) precipitated SRC from RA FLS lysates in pull-down assays, but not YES or FYN (SFKs also expressed in FLS). Additionally, full-length RPTPk immunoprecipitated from COS-1 cells in vitro dephosphorylated a SRC phospho-Y527 peptide, while a catalytically inactive mutant (C1100S ) did not.

In line with previously reported data (Wang, S. E. et al., 2005, Id.), we found no direct effect of RPTPk on TGFβ-mediated signaling in FLS, as assessed by lack of effect of PTPRK ASO on TGFβ1-induced phosphorylation and nuclear recruitment of SMAD3, suggesting that the RA FLS phenotype induced by knockdown of RPTPk is not due to direct inhibition of TGFβ signaling.

### Example 5

### RPTPκ is Required for the Pathogenic Action of RA FLS

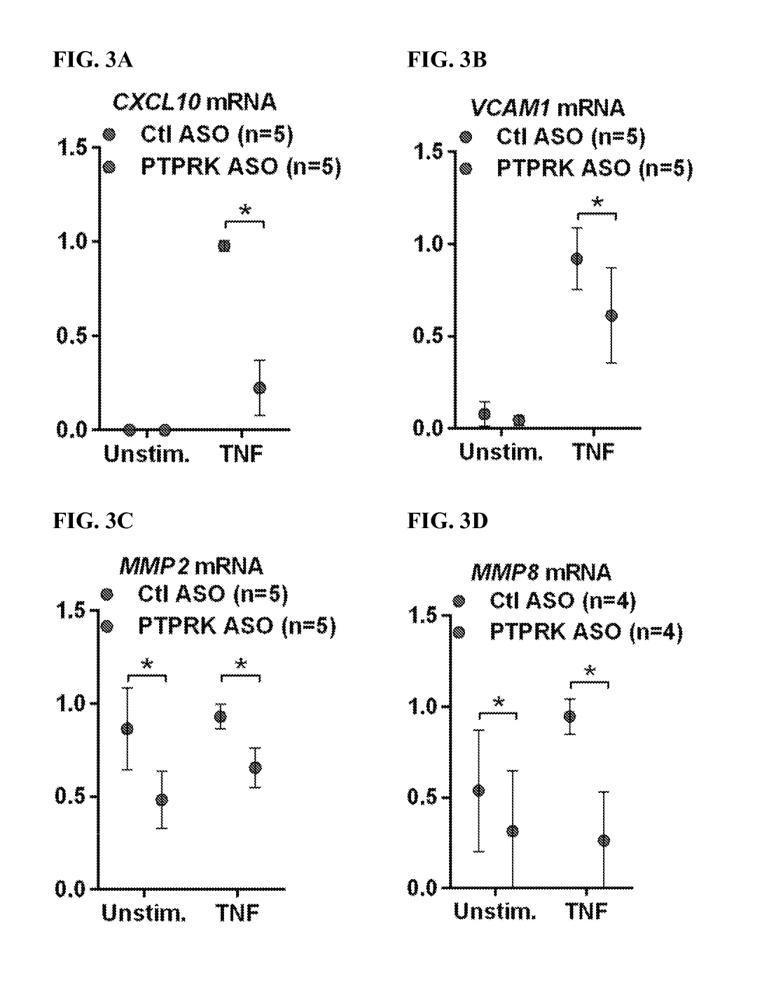

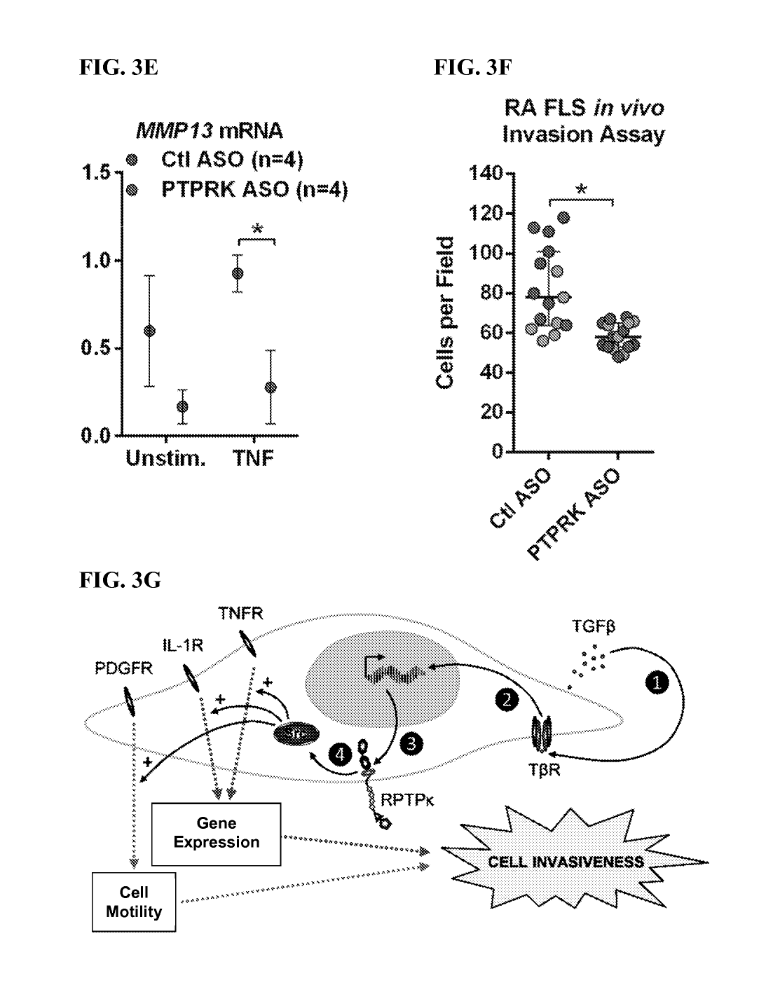

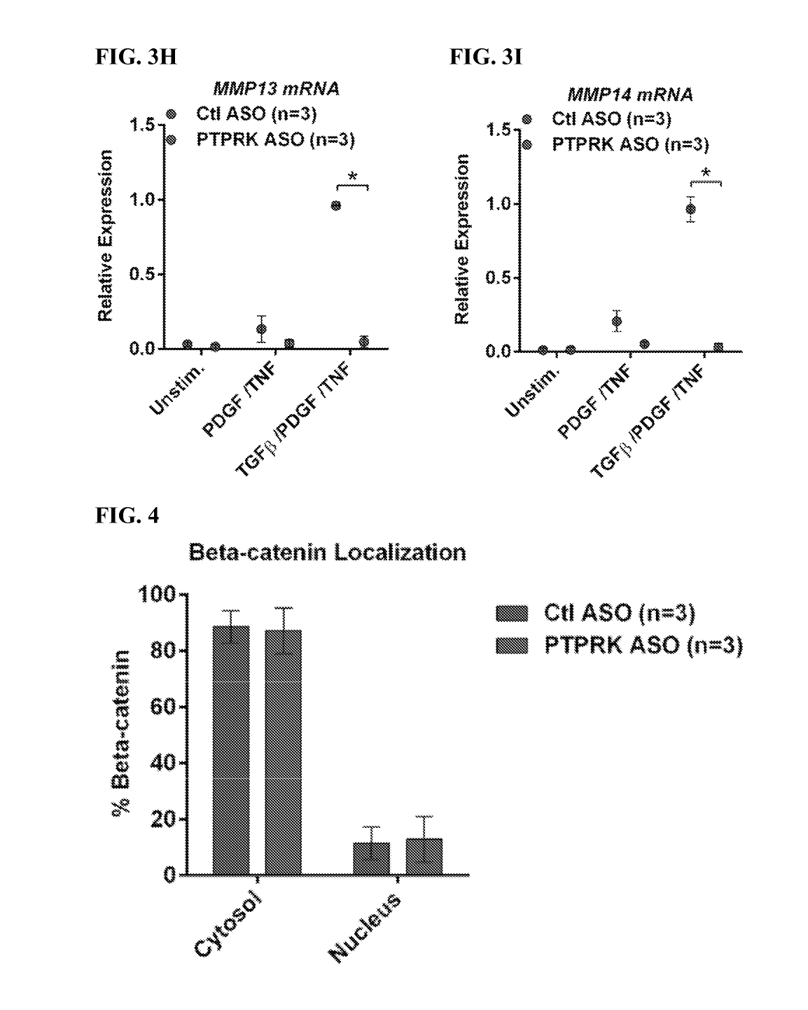

As the rheumatoid synovium is characterized by pathogenic overexpression of TNF and IL-1 (Bottini, N., and Firestein, G. S. 2013, Id.), and SRC activation is known to promote signaling through inflammatory cytokine receptors (Kant, S. et al., 2011. Genes Dev, 25:2069-2078), we assessed the effect of PTPRK ASO on TNF and IL-1-induced expression of genes encoding mediators of FLS invasiveness. RPTPκ deficiency significantly decreased TNF- and IL-1-induced expression of several genes critical for FLS invasion, CXCL10, VCAM1, MMP8 and MMP13 (FIGS. 3A-3E). See e.g., Noss, E. H., & Brenner, M. B., 2008. Immunological reviews, 223:252-270; Bottini, N., and Firestein, G. S. 2013, Id.; Laragione, T. et al., 2011. Arthritis and rheumatism, 63:3274-3283; Seemayer, C. A. et al., 2003. The American journal of pathology, 162:1549-1557. MMP2 expression was not induced by cytokine stimulation but was constitutively decreased following RPTPκ knockdown (FIG. 3C). No effect was observed on the expression of the inflammatory cytokines IL6, IL8, or MMP1 or MMP3.

We then assessed, via Western blotting of lysates from ASO-treated RA FLS stimulated with 50 ng/ml TNFα for 15 min or left stimulated, the effect of PTPRK ASO on TNF-induced activation of mitogen-activated protein kinases (MAPKs) in RA FLS, and found PTPRK ASO reduced basal and TNF-induced phosphorylation of c-Jun N-terminal kinase (JNK), but not ERK or p38. Anti-GAPDH served as control. This phenotype is consistent with a role for RPTPκ upstream the activation of FAK, as FAK promotes downstream activation of JNK and production of MMPs. See e.g., Mitra, S. K., Hanson, D. A., and Schlaepfer, D. D. 2005. Molecular cell biology, 6:56-68.

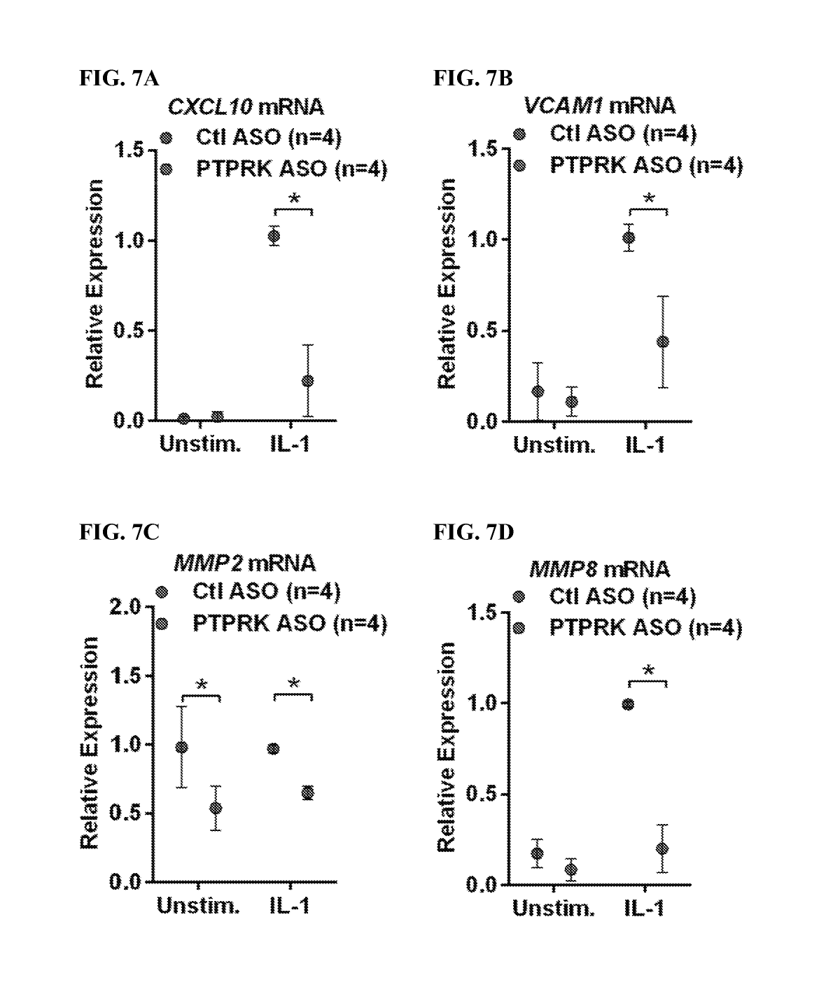

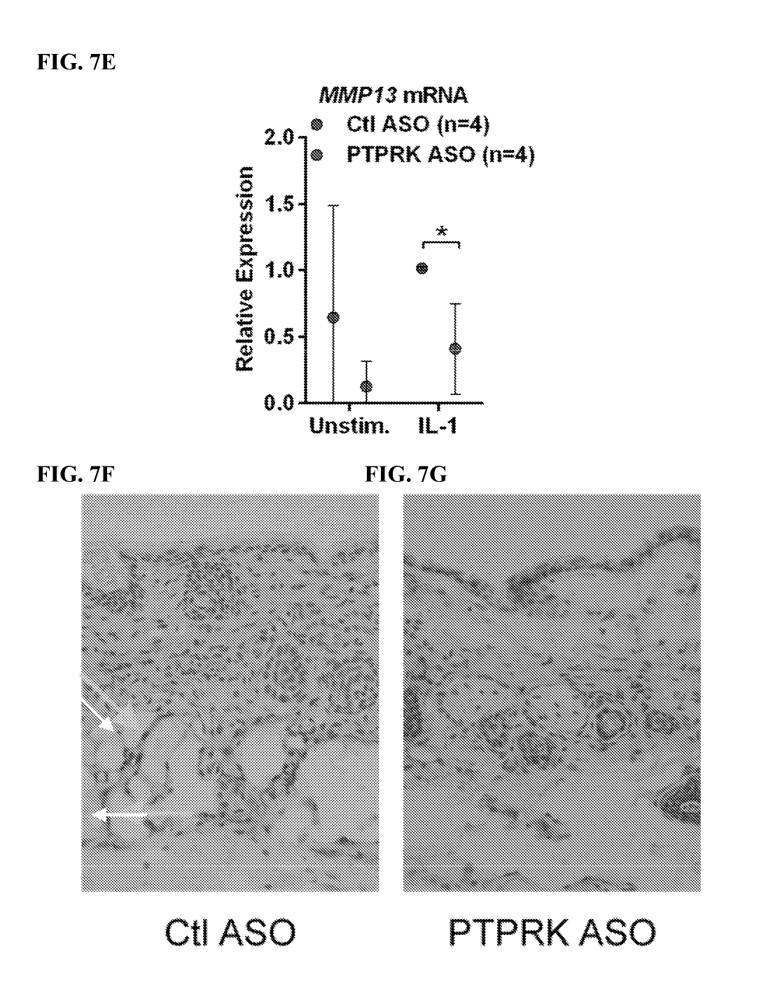

We next examined whether PTPRK ASO inhibits the in vivo invasiveness of RA FLS. We induced skin inflammation in athymic nude mice by subcutaneously injecting complete Freund's adjuvant (CFA), then intradermally implanted RA FLS pretreated with Ctl ASI or PTPRK ASO. After 5 days, we monitored FLS invasion from the implantation site towards the inflammation site. As shown in FIGS. 3F and 7F, PTPRK ASO treatment significantly reduced the in vivo invasiveness of RA FLS.

Without wishing to be bound by any one theory, the data disclosed herein point to a model (FIG. 3G) in which autocrine TGFβ upregulates of RPTPκ expression in RA FLS, in turn leading to increased RA FLS invasiveness through activation of SRC and cross-activation of PDGF and TNF- and IL-1 signaling.

Synergistic stimulation with TGFβ and PDGF strongly amplifies FLS responsiveness to TNF. See e.g., Rosengren, S., Corr, M., and Boyle, D. L. 2010. Arthritis Res Ther, 12:R65). We reasoned that if our model is correct, the contribution of TGFβ to TNF signaling in this system might be mediated through increased expression of PTPRK. We stimulated ASO-treated RA FLS with TGFβ1 for 24 hr to increase PTPRK expression. We then co-stimulated cells with further TGFβ1, and TNF and PDGF for an additional 24 hr. Co-stimulation with TGFβ1 dramatically induced expression of key mediators of RA FLS invasiveness, MMP13 (FIG. 3H) and MMP14 (FIG. 3I), compared to cells lacking stimulation with TGFβ1. Furthermore, treatment with PTPRK ASO completely abolished the TGFβ1-mediated effect.

### CONCLUSION

In this study based entirely on human primary cells from RA patients, we report the first characterization of the role of a transmembrane PTP in RA FLS. We found that RPTPκ is overexpressed in FLS from RA patients compared to OA patients, which results from increased production of TGFβ by these cells. Through dephosphorylation of the inhibitory Y527 of SRC, RPTPκ promotes RA FLS aggressiveness by enhancing responsiveness to PDGF, TNF and IL-1 stimulation. RPTPκ-deficient RA FLS display dramatically reduced spreading, migration, invasiveness and chemokine production. Furthermore, we found that RPTPκ is required for the cross-activation of growth factor and inflammatory cytokine signaling by TGFβ that has been reported to occur in RA FLS.

Our observation that RPTPk promotes RA FLS aggressiveness is surprising compared to previous reports of RPTPk as a tumor suppressor, suggesting that RPTPk controls signaling in RA FLS cancer cells through different mechanisms. PTPRK has been reported to inhibit proliferation of several types of cancer cells, presumably through modulation of beta-catenin function or epidermal growth factor receptor signaling. See e.g., Xu, Y. et al., 2009. J Cell Biochem, 107:873-880; Julien, S. G., Dube, N., Hardy, S., and Tremblay, M. L. 2011. Nat Rev Cancer, 11:35-49. However we found no evidence of a role RPTPk in regulation of beta-catenin function; neither RPTPκ inhibited migration or survival induced by growth factors in RA FLS.

Without further wishing to be bound by any theory, it is believed that in the context of RA, inhibition of RPTPk in RA subjects carrying high expression of PTPRK at the FLS level mitigates the pathogenic effects of FLS. The transmembrane nature of RPTPk suggests potential for modulation through its juxtamembrane or extracellular domains. Indeed inhibition by dimerization has been suggested for other transmembrane PTPs (Majeti, R., and Weiss, A. 2001. Chemical Reviews, 101:2441-2448) and an anti-RPTPk antibody targeted against the extracellular domain was reported to modulate RPTPk activity (Anders, L. et al., 2006. Mol Cell Biol, 26:3917-3934). Deletion of Ptprk in the Long-Evans Cinnamon rat leads to immunodeficiency of T-helper cells (Asano, A. et al., 2007. Mamm Genome, 18:779-786), suggesting inhibition of RPTPk could provide a dual means of affecting RA, targeting both T cell- and FLS-mediated pathogenesis.

### V. Embodiments

Embodiments include embodiments P1 to P15 following.

### Embodiment P1

A method of treating a subject who has or is at risk of developing an autoimmune disease, the method comprising administering to the subject a therapeutically effective amount of an autoimmune therapeutic agent, wherein the autoimmune therapeutic agent is an agonist or an antagonist of PTPRK.

### Embodiment P2

The method of embodiment P1, wherein the autoimmune disease is an inflammatory autoimmune disease and the autoimmune therapeutic agent is an IAD therapeutic agent, the IAD therapeutic agent selected from an anti-PTPRK antibody, an anti-PTPRK inhibitory nucleic acid and a PTPRK ligand mimetic, wherein the IAD therapeutic agent targets PTPRK, or a fragment, agonist or antagonist thereof.

### Embodiment P3

The method embodiment P1 or embodiment P2 wherein the autoimmune therapeutic agent is an antagonist of PTPRK.

### Embodiment P4

The method of embodiment P2, wherein the inflammatory autoimmune disease is mediated by cells expressing PTPRK.

### Embodiment P5

The method of embodiment P4, wherein the cells are fibroblast-like synoviocytes.

### Embodiment P6

The method embodiment P2 or embodiment P3, wherein the inflammatory autoimmune disease is arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma.

### Embodiment P7

The method of one of embodiments P2, P3, or P6, wherein the inflammatory autoimmune disease is rheumatoid arthritis.

### Embodiment P8

The method of any one of embodiments P1 to P7 wherein the method comprises decreasing, reducing, inhibiting, suppressing, limiting or controlling TNF and PDGF activity.

### Embodiment P9

A pharmaceutical composition comprising an autoimmune therapeutic agent and a pharmaceutically acceptable excipient, wherein the autoimmune therapeutic agent is an agonist or antagonist of PTRPK.

### Embodiment P10

The pharmaceutical composition of embodiment P9 wherein the autoimmune therapeutic agent is an antagonist of PTPRK.

### Embodiment P11

The pharmaceutical composition of embodiment P9 or embodiment P10, wherein the pharmaceutical composition comprises an IAD therapeutic agent and a pharmaceutically acceptable excipient, wherein the IAD therapeutic agent is an IAD therapeutic agent selected from an anti-PTPRK antibody, an anti-PTPRK inhibitory nucleic acid or PTPRK ligand mimetic.

### Embodiment P12

The pharmaceutical composition of embodiment P11, wherein the IAD therapeutic agent is an anti-PTPRK antibody.

### Embodiment P13

The pharmaceutical composition of embodiment P11, wherein the IAD therapeutic agent is an anti-PTPRK inhibitory nucleic acid.

### Embodiment P14

The pharmaceutical composition of embodiment P13, wherein the anti-PTPRK inhibitory nucleic acid is an anti-PTPRK antisense nucleic acid.

### Embodiment P15

The pharmaceutical composition of embodiment P11, wherein the PTPRK ligand mimetic is a peptide or a small chemical molecule.

Further embodiments include the following:

### Embodiment 1

A method of treating an autoimmune disease in a subject in need thereof, the method comprising administering to the subject an effective amount of a PTPRK antagonist.

### Embodiment 2

The method of embodiment 1, wherein said autoimmune disease is a fibroblast mediated disease, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

### Embodiment 3

A method of decreasing inflammation in a synovium of a subject in need thereof, the method comprising administering to the subject an effective amount of a PTPRK antagonist.

### Embodiment 4

A method of treating osteoarthritis in a subject in need thereof, the method comprising administering to the subject an effective amount of a PTPRK antagonist.

### Embodiment 5

The method of any one of embodiments 1, 2 or 3, wherein said subject comprises fibroblast-like synoviocytes that express high levels of PTPRK relative to a standard control.

### Embodiment 6

The method of any one of embodiments 3 or 5, wherein said subject has rheumatoid arthritis.

### Embodiment 7

A method of decreasing expression of PTPRK in a fibroblast-like synoviocyte, the method comprising contacting said fibroblast-like synoviocyte with an effective amount of a PTPRK antagonist.

### Embodiment 8

A method of decreasing TNF activity, IL-1 activity or PDGF activity in a fibroblast-like synoviocyte, the method contacting said fibroblast-like synoviocyte with an effective amount of a PTPRK antagonist.

### Embodiment 9

The method of embodiment 8, consisting of decreasing TNF activity or IL-1 activity.

### Embodiment 10

The method of any one of embodiments 8 or 9, wherein said decreasing comprises decreasing expression of TNF or IL-1.

### Embodiment 11

A method of decreasing invasiveness or migration of a fibroblast-like synoviocyte, the method comprising contacting said fibroblast-like synoviocyte with an effective amount of a PTPRK antagonist.

### Embodiment 12

The method of any one embodiments 7 to 11, wherein said fibroblast-like synoviocyte is a rheumatoid arthritis fibroblast-like synoviocyte.

### Embodiment 13

The method of any one embodiments 7 to 12, wherein said fibroblast-like synoviocyte expresses high levels of PTPRK relative to a standard control.

### Embodiment 14

The method of one of embodiments 1 to 13, wherein said PTPRK antagonist is an anti-PTPRK antibody, an anti-PTPRK inhibitory nucleic acid, PTPRK allosteric inhibitor or a PTPRK ligand mimetic.

### Embodiment 15

The method of embodiment 14, wherein said anti-PTPRK antibody is an anti-PTPRK extracellular antibody.

### Embodiment 16

The method of embodiment 14, wherein said anti-PTPRK antibody is an anti-PTPRK dimer inhibiting antibody or a anti-PTPRK dimerizing antibody.

### Embodiment 17

The method of embodiment 14, wherein said anti-PTPRK inhibitory nucleic acid has at least 90% sequence identity to an at least 10 nucleotide contiguous sequence of SEQ ID NO: 1, SEQ ID NO:2 or a complementary sequence thereof.

### Embodiment 18

The method of embodiment 14, wherein said anti-PTPRK ligand mimetic is a peptide or a small chemical molecule.

### Embodiment 19

A pharmaceutical composition comprising a PTPRK antagonist and a pharmaceutically acceptable excipient.

### Embodiment 20

The pharmaceutical composition of embodiment 19, wherein said PTPRK antagonist is an anti-PTPRK antibody, an anti-PTPRK inhibitory nucleic acid or a PTPRK ligand mimetic.

### Embodiment 21

The pharmaceutical composition of embodiment 19, wherein said anti-PTPRK antibody is an anti-PTPRK extracellular antibody.

### Embodiment 22

The pharmaceutical composition of embodiment 19, wherein said anti-PTPRK antibody is an anti-PTPRK dimer inhibiting antibody or an anti-PTPRK dimerizing antibody.

### Embodiment 23

The pharmaceutical composition of embodiment 19, wherein said anti-PTPRK inhibitory nucleic acid has at least 90% sequence identity to an at least 10 nucleotide contiguous sequence of SEQ ID NO: 1, SEQ ID NO:2 or a complementary sequence thereof

### Embodiment 24

The pharmaceutical composition of embodiment 19, wherein said anti-PTPRK ligand mimetic is a peptide or a small chemical molecule.

### VI. Sequences

Homo sapiens protein tyrosine phosphatase, receptor type, K (PTPRK),
transcript variant 1 (NCBI Accession No. NM_001135648.1):
(SEQ ID NO: 1)
GTCTCTCCGGCAGGAGGCGGTGGCGGCGCCGCGCCCGAGCCCGCATTCCTCAGAAGCGCCCGGAGCCCGC
GGGGCGACGTCACCCCCGGCTCCGCCCCCGTCCCTCCCCGAGCAAAGTAACTCTTGACTAACAGGAGCAG
CCTCCGCCGAGCATGGAGAGCTGCCGCCGCGGCCGGCCGGCGACGCTGGCGACGCTTTCGCCCCTGAGGT
AGTTTGGCGACCGCGAAGAAGGAAAAAGGGCGGGCGGGCGGCTGTCCTCTCACCGTCCTCACCCCGCGAG
GCCCGGCCCGCTCCTCCGTCGTGGATTTCGCGGCGATCCCCCCGGCAGCTCTTTGCAAAGCTGCTTGAAA
CTTCTCCCAAACTCGGCATGGATACGACTGCGGCGGCGGCGCTGCCTGCTTTTGTGGCGCTCTTGCTCCT
CTCTCCTTGGCCTCTCCTGGGATCGGCCCAAGGCCAGTTCTCCGCAGGTGGCTGTACTTTTGATGATGGT
CCAGGGGCCTGTGATTACCACCAGGATCTGTATGATGACTTTGAATGGGTGCATGTTAGTGCTCAAGAGC
CTCATTATCTACCACCCGAGATGCCCCAAGGTTCCTATATGATAGTGGACTCTTCAGATCACGACCCTGG
AGAAAAAGCCAGACTTCAGCTGCCTACAATGAAGGAGAACGACACTCACTGCATTGATTTCAGTTACCTA
TTATATAGCCAGAAAGGACTGAATCCTGGCACTTTGAACATATTAGTTAGGGTGAATAAAGGACCTCTTG
CCAATCCAATTTGGAATGTGACTGGATTCACGGGTAGAGATTGGCTTCGGGCTGAGCTAGCAGTGAGCAC
CTTTTGGCCCAATGAATATCAGGTAATATTTGAAGCTGAAGTCTCAGGAGGGAGAAGTGGTTATATTGCC
ATTGATGACATCCAAGTACTGAGTTATCCTTGTGATAAATCTCCTCATTTCCTCCGTCTAGGGGATGTAG
AGGTGAATGCAGGGCAAAACGCTACATTTCAGTGCATTGCCACAGGGAGAGATGCTGTGCATAACAAGTT
ATGGCTCCAGAGACGAAATGGAGAAGATATACCAGTAGCCCAGACTAAGAACATCAATCATAGAAGGTTT
GCCGCTTCCTTCAGATTGCAAGAAGTGACAAAAACTGACCAGGATTTGTATCGCTGTGTAACTCAGTCAG
AACGAGGTTCCGGTGTGTCCAATTTTGCTCAACTTATTGTGAGAGAACCGCCAAGACCCATTGCTCCTCC
TCAGCTTCTTGGTGTTGGGCCTACATATTTGCTGATCCAACTAAATGCCAACTCGATCATTGGCGATGGT
CCTATCATCCTGAAAGAAGTAGAGTACCGAATGACATCAGGATCCTGGACAGAAACCCATGCAGTCAATG
CTCCAACTTACAAATTATGGCATTTAGATCCAGATACCGAATATGAGATCCGAGTTCTACTTACAAGACC
TGGTGAAGGTGGAACGGGGCTCCCAGGACCTCCACTAATCACCAGAACAAAATGTGCAGAACCTATGAGA
ACCCCAAAGACATTAAAGATTGCTGAAATACAGGCAAGACGGATTGCTGTGGACTGGGAATCCTTGGGTT
ACAACATTACGCGTTGCCACACTTTTAATGTCACTATCTGCTACCATTACTTCCGTGGTCACAACGAGAG
CAAGGCAGACTGTTTGGACATGGACCCCAAAGCCCCTCAGCATGTTGTGAACCATCTGCCACCTTATACA
AATGTCAGCCTCAAGATGATCCTAACCAATCCAGAGGGAAGGAAGGAGAGTGAAGAGACAATTATTCAAA
CTGATGAAGATGTGCCTGGTCCCGTACCAGTAAAATCTCTTCAAGGAACATCCTTTGAAAATAAGATCTT
CTTGAACTGGAAAGAACCTTTGGATCCAAATGGAATCATCACTCAATATGAGATCAGCTATAGCAGTATA
AGATCATTTGATCCTGCAGTTCCAGTGGCTGGACCTCCCCAGACTGTATCAAATTTATGGAACAGTACAC
ACCATGTCTTTATGCATCTCCACCCTGGAACCACGTACCAGTTTTTCATAAGAGCCAGCACGGTCAAAGG
CTTTGGTCCAGCCACAGCCATCAATGTCACCACCAATATCTCAGCTCCAACTTTACCTGACTATGAAGGA
GTTGATGCCTCTCTCAATGAAACTGCCACCACAATAACTGTATTGTTGAGACCAGCACAAGCCAAAGGTG
CTCCTATCAGTGCTTATCAGATTGTTGTGGAAGAACTGCACCCACACCGAACCAAGAGAGAAGCCGGAGC
CATGGAATGCTACCAGGTTCCTGTCACATACCAAAATGCCATGAGTGGGGGTGCACCGTATTACTTTGCT
GCAGAACTCCCCCCGGGAAACCTACCTGAGCCTGCCCCGTTCACTGTGGGTGACAATCGGACCTACCAAG
GCTTTTGGAACCCTCCTTTGGCTCCGCGCAAAGGATACAACATCTATTTCCAGGCGATGAGCAGTGTGGA
GAAGGAAACTAAAACCCAGTGCGTACGCATTGCTACAAAAGCAGCAGCAACAGAAGAACCAGAAGTGATC
CCAGATCCCGCCAAGCAGACAGACAGAGTGGTGAAAATAGCAGGAATTAGTGCTGGAATTTTGGTGTTCA
TCCTCCTTCTCCTAGTTGTCATATTAATTGTAAAAAAGAGCAAACTTGCTAAAAAACGCAAAGATGCCAT
GGGGAATACCCGGCAGGAGATGACTCACATGGTGAATGCAATGGATCGAAGTTATGCTGATCAGAGCACT
CTGCATGCAGAAGATCCTCTTTCCATCACCTTCATGGACCAACATAACTTTAGTCCAAGATATGAGAACC
ACAGTGCTACAGCAGAGTCCAGTCGCCTTCTAGACGTACCTCGCTACCTCTGTGAGGGGACGGAATCCCC
TTACCAGACAGGACAGCTGCATCCAGCCATCAGGGTAGCTGATTTACTGCAGCACATTAATCTCATGAAG
ACATCAGACAGCTATGGGTTCAAAGAGGAATATGAGAGCTTTTTTGAAGGACAGTCAGCATCTTGGGATG
TAGCTAAAAAAGATCAAAATAGAGCAAAAAACCGATATGGAAACATTATAGCATATGATCACTCCAGAGT
GATTTTGCAACCCGTAGAGGATGATCCTTCCTCAGATTATATTAATGCCAACTATATTGATATTTGGCTG
TACAGGGATGGCTACCAGAGACCAAGTCATTACATTGCAACCCAAGGTCCCGTTCATGAAACAGTGTATG
ATTTCTGGAGGATGATTTGGCAAGAACAATCTGCTTGCATTGTGATGGTTACAAATTTAGTTGAGGTTGG
CCGGGTTAAATGCTATAAATATTGGCCTGATGATACTGAAGTTTATGGTGACTTCAAAGTAACGTGTGTA
GAAATGGAACCACTTGCTGAATATGTAGTTAGGACATTCACCCTGGAAAGGAGGGGGTACAATGAAATCC
GTGAAGTTAAACAGTTCCATTTCACGGGCTGGCCTGACCATGGAGTGCCCTACCATGCTACAGGGCTGCT
TTCCTTTATCCGGCGAGTCAAGTTATCAAACCCTCCCAGTGCTGGCCCCATCGTTGTACATTGCAGTGCT
GGTGCTGGACGAACTGGCTGCTACATTGTGATTGACATCATGCTAGACATGGCTGAAAGAGAGGGTGTTG
TTGATATTTACAATTGTGTCAAAGCCTTAAGATCTCGGCGTATTAATATGGTCCAGACAGAGGAACAGTA
CATTTTTATTCATGATGCCATTTTAGAAGCCTGCTTATGTGGAGAAACTGCCATACCTGTCTGTGAATTT
AAAGCTGCATATTTTGATATGATTAGAATAGACTCCCAGACTAACTCTTCACATCTCAAGGATGAATTTC
AGACTCTGAATTCAGTCACCCCTCGACTACAAGCTGAAGACTGCAGTATAGCGTGCCTGCCAAGGAACCA
TGACAAGAACCGTTTCATGGACATGCTGCCACCTGACAGATGTCTGCCTTTTTTAATTACAATTGATGGG
GAGAGCAGTAACTACATCAATGCTGCTCTTATGGACAGCTACAGGCAACCAGCTGCTTTCATCGTCACAC
AATACCCTCTGCCAAACACTGTAAAAGACTTCTGGAGATTAGTGTATGATTATGGCTGTACCTCCATTGT
GATGTTAAACGAAGTCGACTTGTCCCAGGGCTGCCCTCAGTACTGGCCAGAGGAAGGGATGCTACGATAT
GGCCCCATCCAAGTGGAATGTATGTCTTGTTCAATGGACTGTGATGTGATCAACCGGATTTTTAGGATAT
GCAATCTAACAAGACCACAGGAAGGTTATCTGATGGTGCAACAGTTTCAGTACCTAGGATGGGCTTCTCA
TCGAGAAGTGCCTGGATCCAAAAGGTCATTCTTGAAACTGATACTTCAGGTGGAAAAGTGGCAGGAGGAA
TGCGAGGAAGGGGAAGGCCGGACGATTATCCACTGCCTAAATGGTGGCGGGCGAAGTGGCATGTTCTGTG
CTATAGGCATCGTTGTTGAAATGGTGAAACGGCAAAATGTTGTCGATGTTTTCCATGCAGTAAAGACACT
GAGGAACAGCAAGCCAAACATGGTGGAAGCCCCGGAGCAATACCGTTTCTGCTATGATGTAGCTTTGGAG
TACCTGGAATCATCTTAGTTGGGTGAGACTCTTTAAAGTGCATCCATGAAGAAACCTGTCCATCTATTGA
GCCAGCAGCTGTTGTACCTGTTACACTTGTGCAGAAAGATTTTAATGTGGGGGGTGGGAGACTTTTACAT
TTGAGAGGTAAAAGTATTTTTTTTATGAAGTTGTGTATCTTAATAAAAAGGACTGAATTAGTTTTTATTA
CTATATTAAAGCATCAACATTTCATGCCACATAAATTATATTTAATAAGAACCAGATTGAAATGAGAACG
TATTGGTGTTTGTACAGTGAACATGCCACCTTTTTTCTCATGGTTTCAGTAGAGCAGCTACCACATGTTG
CATGAGTTCATACTTTCTACGTGGCATTTTTCTCCCTTTCTAAAATGAAAGCTGATGAATCTTAAAAGGA
AGAAGAAAAGAAAAGCTGTGCAAATTCATAGTAAAGTTCGTTTTTTATATGTTTCCAGTGTAGCAGATCT
CTATATAAATATATAAATATATATAACTGGCTTATTTTCTTTTAATGTGCAATGATGGCTGGATCATTTA
AAGTTCTTTTTAGAAAATAACATAAGCCAAAGACTCAAGTGTAAATATGTCTATATGGAGAAAGCACATT
ATATTTATTGGTTACTTACATTCCTTTTTTGATGGCTAAAATACTACCACCACACAATCATCTTTTTTTT
CCTGAAGAAAGCTTTTTCTTTAGCTAAAATCAATTGTAAACGATTTTTGTAGATTATTTTTTGTATGTTT
TAGTGTAAGTAGAAGATAAACTTTTTATTCATAAACCAGGAAGCAATGTTCTTTATAGTGATTCTCTTGT
GTACATGCTTGTGAATTAAATTTGTGTAAAATCCCTTGGCAATTGGGTCTTTTAATATAGGACCAAATTA
AAACATTTTGCTGAATATGTATAGTTTTTCACAATTTCATTAGGTAAATAATGGTTTGGTGATCATACAT
GAGAAATGTACACATTAAAAGGCCTTGCTGACAACTTGCACAATGTTGAACATAGCCTTTAAGCATCATT
TAAATTTTAAAGGAATGGAGTTTTTCAGCCTGTGGCCCAGCACTGGTCAAGAAAACAAGATGGCAACATA
TATGCTTTCAGGGTCAAATTTGAGCAAACTGTAAACTGTCAGGGTGATAAAATGTTTCTCTTGATGTTTA
CATGCACAAGCTTTGCGTTCTGACTATAAAAAGTGTGAACAAATCAATGCCAGATTCCTGTTTTGCGCAT
TGTCATGGGATTCTTAAGTGAACCTTTCTAAATGTGGTCTTGTTCACATGCTCCACGTAGCTGTAACTTC
ACATCATCAGCTTGCAGTTTGTAATTGACTAAAGCATTCCAGTGTCCTCTTTCTAGATTGCCAGCTCATG
ACATGGTGCTTATAAAGATTTAATTAAAGTAAGAATGAAATAAAGTTTTTATAATTATAACAGTTAAAAA
AAAAAAAAAAAAA.
Homo sapiens protein tyrosine phosphatase, receptor type, K (PTPRK),
transcript variant 2 (NCBI Accession NM_002844.3):
(SEQ ID NO: 2)
AGAAGCGCCCGGAGCCCGCGGGGCGACGTCACCCCCGGCTCCGCCCCCGTCCCTCCCCGAGCAAAGTAAC
TCTTGACTAACAGGAGCAGCCTCCGCCGAGCATGGAGAGCTGCCGCCGCGGCCGGCCGGCGACGCTGGCG
ACGCTTTCGCCCCTGAGGTAGTTTGGCGACCGCGAAGAAGGAAAAAGGGCGGGCGGGCGGCTGTCCTCTC
ACCGTCCTCACCCCGCGAGGCCCGGCCCGCTCCTCCGTCGTGGATTTCGCGGCGATCCCCCCGGCAGCTC
TTTGCAAAGCTGCTTGAAACTTCTCCCAAACTCGGCATGGATACGACTGCGGCGGCGGCGCTGCCTGCTT
TTGTGGCGCTCTTGCTCCTCTCTCCTTGGCCTCTCCTGGGATCGGCCCAAGGCCAGTTCTCCGCAGGTGG
CTGTACTTTTGATGATGGTCCAGGGGCCTGTGATTACCACCAGGATCTGTATGATGACTTTGAATGGGTG
CATGTTAGTGCTCAAGAGCCTCATTATCTACCACCCGAGATGCCCCAAGGTTCCTATATGATAGTGGACT
CTTCAGATCACGACCCTGGAGAAAAAGCCAGACTTCAGCTGCCTACAATGAAGGAGAACGACACTCACTG
CATTGATTTCAGTTACCTATTATATAGCCAGAAAGGACTGAATCCTGGCACTTTGAACATATTAGTTAGG
GTGAATAAAGGACCTCTTGCCAATCCAATTTGGAATGTGACTGGATTCACGGGTAGAGATTGGCTTCGGG
CTGAGCTAGCAGTGAGCACCTTTTGGCCCAATGAATATCAGGTAATATTTGAAGCTGAAGTCTCAGGAGG
GAGAAGTGGTTATATTGCCATTGATGACATCCAAGTACTGAGTTATCCTTGTGATAAATCTCCTCATTTC
CTCCGTCTAGGGGATGTAGAGGTGAATGCAGGGCAAAACGCTACATTTCAGTGCATTGCCACAGGGAGAG
ATGCTGTGCATAACAAGTTATGGCTCCAGAGACGAAATGGAGAAGATATACCAGTAGCCCAGACTAAGAA
CATCAATCATAGAAGGTTTGCCGCTTCCTTCAGATTGCAAGAAGTGACAAAAACTGACCAGGATTTGTAT
CGCTGTGTAACTCAGTCAGAACGAGGTTCCGGTGTGTCCAATTTTGCTCAACTTATTGTGAGAGAACCGC
CAAGACCCATTGCTCCTCCTCAGCTTCTTGGTGTTGGGCCTACATATTTGCTGATCCAACTAAATGCCAA
CTCGATCATTGGCGATGGTCCTATCATCCTGAAAGAAGTAGAGTACCGAATGACATCAGGATCCTGGACA
GAAACCCATGCAGTCAATGCTCCAACTTACAAATTATGGCATTTAGATCCAGATACCGAATATGAGATCC
GAGTTCTACTTACAAGACCTGGTGAAGGTGGAACGGGGCTCCCAGGACCTCCACTAATCACCAGAACAAA
ATGTGCAGAACCTATGAGAACCCCAAAGACATTAAAGATTGCTGAAATACAGGCAAGACGGATTGCTGTG
GACTGGGAATCCTTGGGTTACAACATTACGCGTTGCCACACTTTTAATGTCACTATCTGCTACCATTACT
TCCGTGGTCACAACGAGAGCAAGGCAGACTGTTTGGACATGGACCCCAAAGCCCCTCAGCATGTTGTGAA
CCATCTGCCACCTTATACAAATGTCAGCCTCAAGATGATCCTAACCAATCCAGAGGGAAGGAAGGAGAGT
GAAGAGACAATTATTCAAACTGATGAAGATGTGCCTGGTCCCGTACCAGTAAAATCTCTTCAAGGAACAT
CCTTTGAAAATAAGATCTTCTTGAACTGGAAAGAACCTTTGGATCCAAATGGAATCATCACTCAATATGA
GATCAGCTATAGCAGTATAAGATCATTTGATCCTGCAGTTCCAGTGGCTGGACCTCCCCAGACTGTATCA
AATTTATGGAACAGTACACACCATGTCTTTATGCATCTCCACCCTGGAACCACGTACCAGTTTTTCATAA
GAGCCAGCACGGTCAAAGGCTTTGGTCCAGCCACAGCCATCAATGTCACCACCAATATCTCAGCTCCAAC
TTTACCTGACTATGAAGGAGTTGATGCCTCTCTCAATGAAACTGCCACCACAATAACTGTATTGTTGAGA
CCAGCACAAGCCAAAGGTGCTCCTATCAGTGCTTATCAGATTGTTGTGGAAGAACTGCACCCACACCGAA
CCAAGAGAGAAGCCGGAGCCATGGAATGCTACCAGGTTCCTGTCACATACCAAAATGCCATGAGTGGGGG
TGCACCGTATTACTTTGCTGCAGAACTCCCCCCGGGAAACCTACCTGAGCCTGCCCCGTTCACTGTGGGT
GACAATCGGACCTACCAAGGCTTTTGGAACCCTCCTTTGGCTCCGCGCAAAGGATACAACATCTATTTCC
AGGCGATGAGCAGTGTGGAGAAGGAAACTAAAACCCAGTGCGTACGCATTGCTACAAAAGCAGCAGCAAC
AGAAGAACCAGAAGTGATCCCAGATCCCGCCAAGCAGACAGACAGAGTGGTGAAAATAGCAGGAATTAGT
GCTGGAATTTTGGTGTTCATCCTCCTTCTCCTAGTTGTCATATTAATTGTAAAAAAGAGCAAACTTGCTA
AAAAACGCAAAGATGCCATGGGGAATACCCGGCAGGAGATGACTCACATGGTGAATGCAATGGATCGAAG
TTATGCTGATCAGAGCACTCTGCATGCAGAAGATCCTCTTTCCATCACCTTCATGGACCAACATAACTTT
AGTCCAAGATATGAGAACCACAGTGCTACAGCAGAGTCCAGTCGCCTTCTAGACGTACCTCGCTACCTCT
GTGAGGGGACGGAATCCCCTTACCAGACAGGACAGCTGCATCCAGCCATCAGGGTAGCTGATTTACTGCA
GCACATTAATCTCATGAAGACATCAGACAGCTATGGGTTCAAAGAGGAATATGAGAGCTTTTTTGAAGGA
CAGTCAGCATCTTGGGATGTAGCTAAAAAAGATCAAAATAGAGCAAAAAACCGATATGGAAACATTATAG
CATATGATCACTCCAGAGTGATTTTGCAACCCGTAGAGGATGATCCTTCCTCAGATTATATTAATGCCAA
CTATATTGATGGCTACCAGAGACCAAGTCATTACATTGCAACCCAAGGTCCCGTTCATGAAACAGTGTAT
GATTTCTGGAGGATGATTTGGCAAGAACAATCTGCTTGCATTGTGATGGTTACAAATTTAGTTGAGGTTG
GCCGGGTTAAATGCTATAAATATTGGCCTGATGATACTGAAGTTTATGGTGACTTCAAAGTAACGTGTGT
AGAAATGGAACCACTTGCTGAATATGTAGTTAGGACATTCACCCTGGAAAGGAGGGGGTACAATGAAATC
CGTGAAGTTAAACAGTTCCATTTCACGGGCTGGCCTGACCATGGAGTGCCCTACCATGCTACAGGGCTGC
TTTCCTTTATCCGGCGAGTCAAGTTATCAAACCCTCCCAGTGCTGGCCCCATCGTTGTACATTGCAGTGC
TGGTGCTGGACGAACTGGCTGCTACATTGTGATTGACATCATGCTAGACATGGCTGAAAGAGAGGGTGTT
GTTGATATTTACAATTGTGTCAAAGCCTTAAGATCTCGGCGTATTAATATGGTCCAGACAGAGGAACAGT
ACATTTTTATTCATGATGCCATTTTAGAAGCCTGCTTATGTGGAGAAACTGCCATACCTGTCTGTGAATT
TAAAGCTGCATATTTTGATATGATTAGAATAGACTCCCAGACTAACTCTTCACATCTCAAGGATGAATTT
CAGACTCTGAATTCAGTCACCCCTCGACTACAAGCTGAAGACTGCAGTATAGCGTGCCTGCCAAGGAACC
ATGACAAGAACCGTTTCATGGACATGCTGCCACCTGACAGATGTCTGCCTTTTTTAATTACAATTGATGG
GGAGAGCAGTAACTACATCAATGCTGCTCTTATGGACAGCTACAGGCAACCAGCTGCTTTCATCGTCACA
CAATACCCTCTGCCAAACACTGTAAAAGACTTCTGGAGATTAGTGTATGATTATGGCTGTACCTCCATTG
TGATGTTAAACGAAGTCGACTTGTCCCAGGGCTGCCCTCAGTACTGGCCAGAGGAAGGGATGCTACGATA
TGGCCCCATCCAAGTGGAATGTATGTCTTGTTCAATGGACTGTGATGTGATCAACCGGATTTTTAGGATA
TGCAATCTAACAAGACCACAGGAAGGTTATCTGATGGTGCAACAGTTTCAGTACCTAGGATGGGCTTCTC
ATCGAGAAGTGCCTGGATCCAAAAGGTCATTCTTGAAACTGATACTTCAGGTGGAAAAGTGGCAGGAGGA
ATGCGAGGAAGGGGAAGGCCGGACGATTATCCACTGCCTAAATGGTGGCGGGCGAAGTGGCATGTTCTGT
GCTATAGGCATCGTTGTTGAAATGGTGAAACGGCAAAATGTTGTCGATGTTTTCCATGCAGTAAAGACAC
TGAGGAACAGCAAGCCAAACATGGTGGAAGCCCCGGAGCAATACCGTTTCTGCTATGATGTAGCTTTGGA
GTACCTGGAATCATCTTAGTTGGGTGAGACTCTTTAAAGTGCATCCATGAAGAAACCTGTCCATCTATTG
AGCCAGCAGCTGTTGTACCTGTTACACTTGTGCAGAAAGATTTTAATGTGGGGGGTGGGAGACTTTTACA
TTTGAGAGGTAAAAGTATTTTTTTTATGAAGTTGTGTATCTTAATAAAAAGGACTGAATTAGTTTTTATT
ACTATATTAAAGCATCAACATTTCATGCCACATAAATTATATTTAATAAGAACCAGATTGAAATGAGAAC
GTATTGGTGTTTGTACAGTGAACATGCCACCTTTTTTCTCATGGTTTCAGTAGAGCAGCTACCACATGTT
GCATGAGTTCATACTTTCTACGTGGCATTTTTCTCCCTTTCTAAAATGAAAGCTGATGAATCTTAAAAGG
AAGAAGAAAAGAAAAGCTGTGCAAATTCATAGTAAAGTTCGTTTTTTATATGTTTCCAGTGTAGCAGATC
TCTATATAAATATATAAATATATATAACTGGCTTATTTTCTTTTAATGTGCAATGATGGCTGGATCATTT
AAAGTTCTTTTTAGAAAATAACATAAGCCAAAGACTCAAGTGTAAATATGTCTATATGGAGAAAGCACAT
TATATTTATTGGTTACTTACATTCCTTTTTTGATGGCTAAAATACTACCACCACACAATCATCTTTTTTT
TCCTGAAGAAAGCTTTTTCTTTAGCTAAAATCAATTGTAAACGATTTTTGTAGATTATTTTTTGTATGTT
TTAGTGTAAGTAGAAGATAAACTTTTTATTCATAAACCAGGAAGCAATGTTCTTTATAGTGATTCTCTTG
TGTACATGCTTGTGAATTAAATTTGTGTAAAATCCCTTGGCAATTGGGTCTTTTAATATAGGACCAAATT
AAAACATTTTGCTGAATATGTATAGTTTTTCACAATTTCATTAGGTAAATAATGGTTTGGTGATCATACA
TGAGAAATGTACACATTAAAAGGCCTTGCTGACAACTTGCACAATGTTGAACATAGCCTTTAAGCATCAT
TTAAATTTTAAAGGAATGGAGTTTTTCAGCCTGTGGCCCAGCACTGGTCAAGAAAACAAGATGGCAACAT
ATATGCTTTCAGGGTCAAATTTGAGCAAACTGTAAACTGTCAGGGTGATAAAATGTTTCTCTTGATGTTT
ACATGCACAAGCTTTGCGTTCTGACTATAAAAAGTGTGAACAAATCAATGCCAGATTCCTGTTTTGCGCA
TTGTCATGGGATTCTTAAGTGAACCTTTCTAAATGTGGTCTTGTTCACATGCTCCACGTAGCTGTAACTT
CACATCATCAGCTTGCAGTTTGTAATTGACTAAAGCATTCCAGTGTCCTCTTTCTAGATTGCCAGCTCAT
GACATGGTGCTTATAAAGATTTAATTAAAGTAAGAATGAAATAAAGTTTTTATAATTATAACAGTTAAAA
AAAAAAAAAAAAAA.
PTPRK ASO:
(SEQ ID NO: 3)
TCTTAATCACAACCTACCACAAGGA.
PTPRK_2 ASO:
(SEQ ID NO: 4)
ACAGCAAAGTATGAGCATACCATCT.
PTPRM ASO:
(SEQ ID NO: 5)
TAAAGACAACTTACTACATGGATGT.
Control ASO:
(SEQ ID NO: 6)
CCTCTTACCTCAGTTACAATTTATA.

1. (canceled)

2. (canceled)

3. A method of decreasing inflammation in a synovium of a subject in need thereof, the method comprising administering to the subject an effective amount of a PTPRK antagonist.

4. A method of treating osteoarthritis in a subject in need thereof, the method comprising administering to the subject an effective amount of a PTPRK antagonist.

5. The method of claim 3, wherein said subject comprises fibroblast-like synoviocytes that express high levels of PTPRK relative to a standard control.

6. The method of claim 5, wherein said subject has rheumatoid arthritis.

7. (canceled)

8. (canceled)

9. (canceled)

10. (canceled)

11. A method of decreasing invasiveness or migration of a fibroblast-like synoviocyte in a subject, the method comprising contacting said fibroblast-like synoviocyte in a subject in need thereof with an effective amount of a PTPRK antagonist.

12. The method of any claim 11, wherein said fibroblast-like synoviocyte is a rheumatoid arthritis fibroblast-like synoviocyte.

13. The method of claim 12, wherein said fibroblast-like synoviocyte expresses high levels of PTPRK relative to a standard control.

14. The method of claim 13, wherein said PTPRK antagonist is an anti-PTPRK antibody, an anti-PTPRK inhibitory nucleic acid, PTPRK allosteric inhibitor or a PTPRK ligand mimetic.

15. The method of claim 14, wherein said anti-PTPRK antibody is an anti-PTPRK extracellular antibody.

16. The method of claim 14, wherein said anti-PTPRK antibody is an anti-PTPRK dimer inhibiting antibody or an anti-PTPRK dimerizing antibody.

17. The method of claim 14, wherein said anti-PTPRK inhibitory nucleic acid has at least 90% sequence identity to an at least 10 nucleotide contiguous sequence of SEQ ID NO:1, SEQ ID NO:2 or a complementary sequence thereof.

18. The method of claim 14, wherein said anti-PTPRK ligand mimetic is a peptide or a small chemical molecule.

19. (canceled)

20. (canceled)

21. (canceled)

22. (canceled)

23. (canceled)

24. (canceled)

25. The method of claim 3, further comprising treating an autoimmune disease in said subject.

26. The method of claim 25, wherein said autoimmune disease is a fibroblast mediated disease, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

27. The method of claim 5, wherein said PTPRK antagonist decreases TNF activity, IL-1 activity or PDGF activity in said fibroblast-like synoviocyte.

28. The method of claim 11, further comprising treating an autoimmune disease in said subject.

29. The method of claim 28 wherein said autoimmune disease is fibroblast mediated disease, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, scleroderma, systemic sclerosis, or allergic asthma.

30. The method of claim 11, wherein said PTPRK antagonist decreases TNF activity, IL-1 activity or PDGF activity in said fibroblast-like synoviocyte.

31. The method of claim 11, wherein said PTPRK antagonist is an anti-PTPRK antibody, an anti-PTPRK inhibitory nucleic acid, PTPRK allosteric inhibitor or a PTPRK ligand mimetic.

32. The method of claim 4, wherein said PTPRK antagonist is an anti-PTPRK antibody, an anti-PTPRK inhibitory nucleic acid, PTPRK allosteric inhibitor or a PTPRK ligand mimetic.


SEQUENCE LISTING (6 sequences)

SEQ ID NO: 1 - DNA, 6173 bp, Homo sapiens
gtctctccggcaggaggcggtggcggcgccgcgcccgagcccgcattcctcagaagcgcccggagcccgcggggcgacgtcacccccggctccgcccccgtccctccccgagcaaagtaactcttgactaacaggagcagcctccgccgagcatggagagctgccgccgcggccggccggcgacgctggcgacgctttcgcccctgaggtagtttggcgaccgcgaagaaggaaaaagggcgggcgggcggctgtcctctcaccgtcctcaccccgcgaggcccggcccgctcctccgtcgtggatttcgcggcgatccccccggcagctctttgcaaagctgcttgaaacttctcccaaactcggcatggatacgactgcggcggcggcgctgcctgcttttgtggcgctcttgctcctctctccttggcctctcctgggatcggcccaaggccagttctccgcaggtggctgtacttttgatgatggtccaggggcctgtgattaccaccaggatctgtatgatgactttgaatgggtgcatgttagtgctcaagagcctcattatctaccacccgagatgccccaaggttcctatatgatagtggactcttcagatcacgaccctggagaaaaagccagacttcagctgcctacaatgaaggagaacgacactcactgcattgatttcagttacctattatatagccagaaaggactgaatcctggcactttgaacatattagttagggtgaataaaggacctcttgccaatccaatttggaatgtgactggattcacgggtagagattggcttcgggctgagctagcagtgagcaccttttggcccaatgaatatcaggtaatatttgaagctgaagtctcaggagggagaagtggttatattgccattgatgacatccaagtactgagttatccttgtgataaatctcctcatttcctccgtctaggggatgtagaggtgaatgcagggcaaaacgctacatttcagtgcattgccacagggagagatgctgtgcataacaagttatggctccagagacgaaatggagaagatataccagtagcccagactaagaacatcaatcatagaaggtttgccgcttccttcagattgcaagaagtgacaaaaactgaccaggatttgtatcgctgtgtaactcagtcagaacgaggttccggtgtgtccaattttgctcaacttattgtgagagaaccgccaagacccattgctcctcctcagcttcttggtgttgggcctacatatttgctgatccaactaaatgccaactcgatcattggcgatggtcctatcatcctgaaagaagtagagtaccgaatgacatcaggatcctggacagaaacccatgcagtcaatgctccaacttacaaattatggcatttagatccagataccgaatatgagatccgagttctacttacaagacctggtgaaggtggaacggggctcccaggacctccactaatcaccagaacaaaatgtgcagaacctatgagaaccccaaagacattaaagattgctgaaatacaggcaagacggattgctgtggactgggaatccttgggttacaacattacgcgttgccacacttttaatgtcactatctgctaccattacttccgtggtcacaacgagagcaaggcagactgtttggacatggaccccaaagcccctcagcatgttgtgaaccatctgccaccttatacaaatgtcagcctcaagatgatcctaaccaatccagagggaaggaaggagagtgaagagacaattattcaaactgatgaagatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactccccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggaatacccggcaggagatgactcacatggtgaatgcaatggatcgaagttatgctgatcagagcactctgcatgcagaagatcctctttccatcaccttcatggaccaacataactttagtccaagatatgagaaccacagtgctacagcagagtccagtcgccttctagacgtacctcgctacctctgtgaggggacggaatccccttaccagacaggacagctgcatccagccatcagggtagctgatttactgcagcacattaatctcatgaagacatcagacagctatgggttcaaagaggaatatgagagcttttttgaaggacagtcagcatcttgggatgtagctaaaaaagatcaaaatagagcaaaaaaccgatatggaaacattatagcatatgatcactccagagtgattttgcaacccgtagaggatgatccttcctcagattatattaatgccaactatattgatatttggctgtacagggatggctaccagagaccaagtcattacattgcaacccaaggtcccgttcatgaaacagtgtatgatttctggaggatgatttggcaagaacaatctgcttgcattgtgatggttacaaatttagttgaggttggccgggttaaatgctataaatattggcctgatgatactgaagtttatggtgacttcaaagtaacgtgtgtagaaatggaaccacttgctgaatatgtagttaggacattcaccctggaaaggagggggtacaatgaaatccgtgaagttaaacagttccatttcacgggctggcctgaccatggagtgccctaccatgctacagggctgctttcctttatccggcgagtcaagttatcaaaccctcccagtgctggccccatcgttgtacattgcagtgctggtgctggacgaactggctgctacattgtgattgacatcatgctagacatggctgaaagagagggtgttgttgatatttacaattgtgtcaaagccttaagatctcggcgtattaatatggtccagacagaggaacagtacatttttattcatgatgccattttagaagcctgcttatgtggagaaactgccatacctgtctgtgaatttaaagctgcatattttgatatgattagaatagactcccagactaactcttcacatctcaaggatgaatttcagactctgaattcagtcacccctcgactacaagctgaagactgcagtatagcgtgcctgccaaggaaccatgacaagaaccgtttcatggacatgctgccacctgacagatgtctgccttttttaattacaattgatggggagagcagtaactacatcaatgctgctcttatggacagctacaggcaaccagctgctttcatcgtcacacaataccctctgccaaacactgtaaaagacttctggagattagtgtatgattatggctgtacctccattgtgatgttaaacgaagtcgacttgtcccagggctgccctcagtactggccagaggaagggatgctacgatatggccccatccaagtggaatgtatgtcttgttcaatggactgtgatgtgatcaaccggatttttaggatatgcaatctaacaagaccacaggaaggttatctgatggtgcaacagtttcagtacctaggatgggcttctcatcgagaagtgcctggatccaaaaggtcattcttgaaactgatacttcaggtggaaaagtggcaggaggaatgcgaggaaggggaaggccggacgattatccactgcctaaatggtggcgggcgaagtggcatgttctgtgctataggcatcgttgttgaaatggtgaaacggcaaaatgttgtcgatgttttccatgcagtaaagacactgaggaacagcaagccaaacatggtggaagccccggagcaataccgtttctgctatgatgtagctttggagtacctggaatcatcttagttgggtgagactctttaaagtgcatccatgaagaaacctgtccatctattgagccagcagctgttgtacctgttacacttgtgcagaaagattttaatgtggggggtgggagacttttacatttgagaggtaaaagtattttttttatgaagttgtgtatcttaataaaaaggactgaattagtttttattactatattaaagcatcaacatttcatgccacataaattatatttaataagaaccagattgaaatgagaacgtattggtgtttgtacagtgaacatgccaccttttttctcatggtttcagtagagcagctaccacatgttgcatgagttcatactttctacgtggcatttttctccctttctaaaatgaaagctgatgaatcttaaaaggaagaagaaaagaaaagctgtgcaaattcatagtaaagttcgttttttatatgtttccagtgtagcagatctctatataaatatataaatatatataactggcttattttcttttaatgtgcaatgatggctggatcatttaaagttctttttagaaaataacataagccaaagactcaagtgtaaatatgtctatatggagaaagcacattatatttattggttacttacattccttttttgatggctaaaatactaccaccacacaatcatcttttttttcctgaagaaagctttttctttagctaaaatcaattgtaaacgatttttgtagattattttttgtatgttttagtgtaagtagaagataaactttttattcataaaccaggaagcaatgttctttatagtgattctcttgtgtacatgcttgtgaattaaatttgtgtaaaatcccttggcaattgggtcttttaatataggaccaaattaaaacattttgctgaatatgtatagtttttcacaatttcattaggtaaataatggtttggtgatcatacatgagaaatgtacacattaaaaggccttgctgacaacttgcacaatgttgaacatagcctttaagcatcatttaaattttaaaggaatggagtttttcagcctgtggcccagcactggtcaagaaaacaagatggcaacatatatgctttcagggtcaaatttgagcaaactgtaaactgtcagggtgataaaatgtttctcttgatgtttacatgcacaagctttgcgttctgactataaaaagtgtgaacaaatcaatgccagattcctgttttgcgcattgtcatgggattcttaagtgaacctttctaaatgtggtcttgttcacatgctccacgtagctgtaacttcacatcatcagcttgcagtttgtaattgactaaagcattccagtgtcctctttctagattgccagctcatgacatggtgcttataaagatttaattaaagtaagaatgaaataaagtttttataattataacagttaaaaaaaaaaaaaaaaaa

SEQ ID NO: 2 - DNA, 6104 bp, Homo sapiens
agaagcgcccggagcccgcggggcgacgtcacccccggctccgcccccgtccctccccgagcaaagtaactcttgactaacaggagcagcctccgccgagcatggagagctgccgccgcggccggccggcgacgctggcgacgctttcgcccctgaggtagtttggcgaccgcgaagaaggaaaaagggcgggcgggcggctgtcctctcaccgtcctcaccccgcgaggcccggcccgctcctccgtcgtggatttcgcggcgatccccccggcagctctttgcaaagctgcttgaaacttctcccaaactcggcatggatacgactgcggcggcggcgctgcctgcttttgtggcgctcttgctcctctctccttggcctctcctgggatcggcccaaggccagttctccgcaggtggctgtacttttgatgatggtccaggggcctgtgattaccaccaggatctgtatgatgactttgaatgggtgcatgttagtgctcaagagcctcattatctaccacccgagatgccccaaggttcctatatgatagtggactcttcagatcacgaccctggagaaaaagccagacttcagctgcctacaatgaaggagaacgacactcactgcattgatttcagttacctattatatagccagaaaggactgaatcctggcactttgaacatattagttagggtgaataaaggacctcttgccaatccaatttggaatgtgactggattcacgggtagagattggcttcgggctgagctagcagtgagcaccttttggcccaatgaatatcaggtaatatttgaagctgaagtctcaggagggagaagtggttatattgccattgatgacatccaagtactgagttatccttgtgataaatctcctcatttcctccgtctaggggatgtagaggtgaatgcagggcaaaacgctacatttcagtgcattgccacagggagagatgctgtgcataacaagttatggctccagagacgaaatggagaagatataccagtagcccagactaagaacatcaatcatagaaggtttgccgcttccttcagattgcaagaagtgacaaaaactgaccaggatttgtatcgctgtgtaactcagtcagaacgaggttccggtgtgtccaattttgctcaacttattgtgagagaaccgccaagacccattgctcctcctcagcttcttggtgttgggcctacatatttgctgatccaactaaatgccaactcgatcattggcgatggtcctatcatcctgaaagaagtagagtaccgaatgacatcaggatcctggacagaaacccatgcagtcaatgctccaacttacaaattatggcatttagatccagataccgaatatgagatccgagttctacttacaagacctggtgaaggtggaacggggctcccaggacctccactaatcaccagaacaaaatgtgcagaacctatgagaaccccaaagacattaaagattgctgaaatacaggcaagacggattgctgtggactgggaatccttgggttacaacattacgcgttgccacacttttaatgtcactatctgctaccattacttccgtggtcacaacgagagcaaggcagactgtttggacatggaccccaaagcccctcagcatgttgtgaaccatctgccaccttatacaaatgtcagcctcaagatgatcctaaccaatccagagggaaggaaggagagtgaagagacaattattcaaactgatgaagatgtgcctggtcccgtaccagtaaaatctcttcaaggaacatcctttgaaaataagatcttcttgaactggaaagaacctttggatccaaatggaatcatcactcaatatgagatcagctatagcagtataagatcatttgatcctgcagttccagtggctggacctccccagactgtatcaaatttatggaacagtacacaccatgtctttatgcatctccaccctggaaccacgtaccagtttttcataagagccagcacggtcaaaggctttggtccagccacagccatcaatgtcaccaccaatatctcagctccaactttacctgactatgaaggagttgatgcctctctcaatgaaactgccaccacaataactgtattgttgagaccagcacaagccaaaggtgctcctatcagtgcttatcagattgttgtggaagaactgcacccacaccgaaccaagagagaagccggagccatggaatgctaccaggttcctgtcacataccaaaatgccatgagtgggggtgcaccgtattactttgctgcagaactccccccgggaaacctacctgagcctgccccgttcactgtgggtgacaatcggacctaccaaggcttttggaaccctcctttggctccgcgcaaaggatacaacatctatttccaggcgatgagcagtgtggagaaggaaactaaaacccagtgcgtacgcattgctacaaaagcagcagcaacagaagaaccagaagtgatcccagatcccgccaagcagacagacagagtggtgaaaatagcaggaattagtgctggaattttggtgttcatcctccttctcctagttgtcatattaattgtaaaaaagagcaaacttgctaaaaaacgcaaagatgccatggggaatacccggcaggagatgactcacatggtgaatgcaatggatcgaagttatgctgatcagagcactctgcatgcagaagatcctctttccatcaccttcatggaccaacataactttagtccaagatatgagaaccacagtgctacagcagagtccagtcgccttctagacgtacctcgctacctctgtgaggggacggaatccccttaccagacaggacagctgcatccagccatcagggtagctgatttactgcagcacattaatctcatgaagacatcagacagctatgggttcaaagaggaatatgagagcttttttgaaggacagtcagcatcttgggatgtagctaaaaaagatcaaaatagagcaaaaaaccgatatggaaacattatagcatatgatcactccagagtgattttgcaacccgtagaggatgatccttcctcagattatattaatgccaactatattgatggctaccagagaccaagtcattacattgcaacccaaggtcccgttcatgaaacagtgtatgatttctggaggatgatttggcaagaacaatctgcttgcattgtgatggttacaaatttagttgaggttggccgggttaaatgctataaatattggcctgatgatactgaagtttatggtgacttcaaagtaacgtgtgtagaaatggaaccacttgctgaatatgtagttaggacattcaccctggaaaggagggggtacaatgaaatccgtgaagttaaacagttccatttcacgggctggcctgaccatggagtgccctaccatgctacagggctgctttcctttatccggcgagtcaagttatcaaaccctcccagtgctggccccatcgttgtacattgcagtgctggtgctggacgaactggctgctacattgtgattgacatcatgctagacatggctgaaagagagggtgttgttgatatttacaattgtgtcaaagccttaagatctcggcgtattaatatggtccagacagaggaacagtacatttttattcatgatgccattttagaagcctgcttatgtggagaaactgccatacctgtctgtgaatttaaagctgcatattttgatatgattagaatagactcccagactaactcttcacatctcaaggatgaatttcagactctgaattcagtcacccctcgactacaagctgaagactgcagtatagcgtgcctgccaaggaaccatgacaagaaccgtttcatggacatgctgccacctgacagatgtctgccttttttaattacaattgatggggagagcagtaactacatcaatgctgctcttatggacagctacaggcaaccagctgctttcatcgtcacacaataccctctgccaaacactgtaaaagacttctggagattagtgtatgattatggctgtacctccattgtgatgttaaacgaagtcgacttgtcccagggctgccctcagtactggccagaggaagggatgctacgatatggccccatccaagtggaatgtatgtcttgttcaatggactgtgatgtgatcaaccggatttttaggatatgcaatctaacaagaccacaggaaggttatctgatggtgcaacagtttcagtacctaggatgggcttctcatcgagaagtgcctggatccaaaaggtcattcttgaaactgatacttcaggtggaaaagtggcaggaggaatgcgaggaaggggaaggccggacgattatccactgcctaaatggtggcgggcgaagtggcatgttctgtgctataggcatcgttgttgaaatggtgaaacggcaaaatgttgtcgatgttttccatgcagtaaagacactgaggaacagcaagccaaacatggtggaagccccggagcaataccgtttctgctatgatgtagctttggagtacctggaatcatcttagttgggtgagactctttaaagtgcatccatgaagaaacctgtccatctattgagccagcagctgttgtacctgttacacttgtgcagaaagattttaatgtggggggtgggagacttttacatttgagaggtaaaagtattttttttatgaagttgtgtatcttaataaaaaggactgaattagtttttattactatattaaagcatcaacatttcatgccacataaattatatttaataagaaccagattgaaatgagaacgtattggtgtttgtacagtgaacatgccaccttttttctcatggtttcagtagagcagctaccacatgttgcatgagttcatactttctacgtggcatttttctccctttctaaaatgaaagctgatgaatcttaaaaggaagaagaaaagaaaagctgtgcaaattcatagtaaagttcgttttttatatgtttccagtgtagcagatctctatataaatatataaatatatataactggcttattttcttttaatgtgcaatgatggctggatcatttaaagttctttttagaaaataacataagccaaagactcaagtgtaaatatgtctatatggagaaagcacattatatttattggttacttacattccttttttgatggctaaaatactaccaccacacaatcatcttttttttcctgaagaaagctttttctttagctaaaatcaattgtaaacgatttttgtagattattttttgtatgttttagtgtaagtagaagataaactttttattcataaaccaggaagcaatgttctttatagtgattctcttgtgtacatgcttgtgaattaaatttgtgtaaaatcccttggcaattgggtcttttaatataggaccaaattaaaacattttgctgaatatgtatagtttttcacaatttcattaggtaaataatggtttggtgatcatacatgagaaatgtacacattaaaaggccttgctgacaacttgcacaatgttgaacatagcctttaagcatcatttaaattttaaaggaatggagtttttcagcctgtggcccagcactggtcaagaaaacaagatggcaacatatatgctttcagggtcaaatttgagcaaactgtaaactgtcagggtgataaaatgtttctcttgatgtttacatgcacaagctttgcgttctgactataaaaagtgtgaacaaatcaatgccagattcctgttttgcgcattgtcatgggattcttaagtgaacctttctaaatgtggtcttgttcacatgctccacgtagctgtaacttcacatcatcagcttgcagtttgtaattgactaaagcattccagtgtcctctttctagattgccagctcatgacatggtgcttataaagatttaattaaagtaagaatgaaataaagtttttataattataacagttaaaaaaaaaaaaaaaaaa

SEQ ID NO: 3 - DNA, 25 bp, Artificial Sequence
  Synthetic polynucleotide
tcttaatcacaacctaccacaagga

SEQ ID NO: 4 - DNA, 25 bp, Artificial Sequence
  Synthetic polynucleotide
acagcaaagtatgagcataccatct

SEQ ID NO: 5 - DNA, 25 bp, Artificial Sequence
  Synthetic polynucleotide
taaagacaacttactacatggatgt

SEQ ID NO: 6 - DNA, 25 bp, Artificial Sequence
  Synthetic polynucleotide
cctcttacctcagttacaatttata